(19) United States
(12) Patent Application Publication
(10) Pub. No.: US 2026/0090921 A1
(43) Pub. Date: Apr. 2, 2026

(54) METHOD FOR FORMING A CELLULOSIC TAMPON APPLICATOR

(71) Applicant: THE PROCTER & GAMBLE COMPANY, Cincinnati, OH (US)

(72) Inventors: Robert Paul CASSONI, Waynesville, OH (US); Benjamin Jacob CLARE, Cincinnati, OH (US); Gavin John BROAD, Fairfield Township, OH (US); Edward Daniel THEISS, III, Cincinnati, OH (US); David Christopher RAGNACCI, JR., West Chester, OH (US)

(21) Appl. No.: 19/345,671

(22) Filed: Sep. 30, 2025

Related U.S. Application Data
(60) Provisional application No. 63/701,863, filed on Oct. 1, 2024.

(51) Int. Cl. A61F 13/20

(52) U.S. Cl. CPC A61F 13/2097; A61F 13/2051

(57) ABSTRACT

A tampon product and method are presented that includes an applicator including an outer member and an inner member slidably engaged with the outer member. The outer member includes an outer surface surrounding a longitudinal axis and a grip region, an intermediate region, and an insertion region. The grip region includes an inverted region such that a portion of a wall of the grip end region is adjacent to the inner surface of the cylindrical paper segment, and a pusher opening internal to the cylindrical paper segment.

### CROSS REFERENCE TO RELATED APPLICATIONS

This application claims the benefit, under 35 U.S.C. § 119 (c), to U.S. Provisional Application No. 63/701,863, filed Oct. 1, 2024, the entire disclosure of which is fully incorporated by reference herein.

### FIELD

The present disclosure relates to tampon applicators, and more particularly, to a method for forming tampon applicators made of cellulosic material.

### BACKGROUND

Consumer products or portions thereof that have components derived from petroleum (such as but not limited to plastics), are currently becoming subject to increased regulation in a number of countries, and growing disfavor in some segments of the consumer market. Some reasons for this relate generally to the consumer perception of certain materials having a relatively adverse impact on the environment.

Tampons have been used for many years to manage menstrual discharge. Many consumers prefer tampons provided inside disposable applicators (rather than tampons configured to be inserted via a finger (“digitally”) without use of an applicator). The typical tampon applicator is configured to house and protect the new tampon absorbent material from deformation and/or contamination prior to use, and then to facilitate application of the tampon when its use is desired.

Currently, many tampon applicators or parts thereof are formed of thermoplastics such as polyethylene. The properties of thermoplastics and known associated forming/molding technologies enable a manufacturer to efficiently make an applicator having relatively sophisticated features, which may be provided for purposes of functionality, convenience, comfort during use, ergonomics and/or aesthetic appeal, thereby making it attractive and convenient to consumers. FIG. 1A depicts a conventional plastic applicator 10 with a barrel region 12 having an external diameter 13, a pusher 30 having an external diameter 31, and a grip region 18 positioned between the barrel region 12 and pusher 30. As shown in FIG. 1A, the external diameter 31 of the pusher 30 is noticeably less than the external diameter 13 of the barrel region 12.

Other types of tampon applicators currently marketed are formed of paperboard or cardboard. While such applicators reduce reliance on and use of materials derived from petroleum as compared to plastic applicators, currently, these cardboard applicators do not provide the same functionality, comfort during use, ergonomics, and/or aesthetic appeal as plastic applicators. FIG. 1B depicts a conventional cardboard applicator 10′ including a barrel region 12′ having an external diameter 13′, a pusher 30′ having an external diameter 31′ and a grip region 18′ between the barrel region 12′ and the pusher 30′. Unlike the plastic applicator 10 of FIG. 1A, the external diameter 31′ of the pusher 30′ is relatively close the external diameter 13′ of the barrel region 12′. FIG. 1C depicts another conventional cardboard applicator 10′ where the grip region 18′ includes four impressions which are spaced apart by the outer surface 42′ of the grip region 18′.

Accordingly, there remain opportunities for improvements in cardboard applicators design and functionality such that carboard applicators more closely emulate their plastic applicator counterparts.

### SUMMARY

The inventors of the present invention recognized various advantages of the conventional plastic applicator that are absent in conventional cardboard applicators. These include features of the plastic applicators which facilitate functionality, convenience, comfort during use, ergonomics and/or aesthetic appeal. The inventors also recognized that recent environmental regulations in various geographical territories that promote the use of cardboard material over plastic material will likely cause a shift in consumer demand towards cardboard applicators. Thus, the inventors developed an improved cardboard applicator herein, which achieves many of the advantages of plastic applicators that are absent in conventional cardboard applicators. In order to achieve this improved cardboard applicator, the inventors necessarily overcame various design obstacles not encountered in manufacturing plastic applicators.

In some embodiments, a method of forming a tampon applicator includes: providing a cylindrical paper segment having a longitudinal axis, an outer surface and an inner surface surrounding the longitudinal axis, wherein the leading edge portion or the trailing edge portion forms a portion of the outer surface comprising a seam; inserting a forming rod into the cylindrical paper segment along the longitudinal axis; and inverting a first end region of the cylindrical paper segment such that a portion of a wall of the first end region is adjacent to an inner surface of the cylindrical paper segment, wherein the edge of the first end region is formed about the forming rod to form a pusher opening.

In some embodiments, a method of forming a tampon applicator includes: providing a cylindrical paper segment having a longitudinal axis, an outer surface and an inner surface surrounding the longitudinal axis, wherein the leading edge portion or the trailing edge portion forms a portion of the outer surface comprising a scam; inserting a first mandrel into a portion of the cylindrical paper segment; inserting a forming rod into a portion of the cylindrical paper segment along the longitudinal axis; engaging a first end region with the first mandrel; and inverting a portion of the first end region of the cylindrical paper segment such that a portion of the first end region is adjacent to an inner surface of the cylindrical paper segment, wherein the edge of the first end region is formed about the forming rod to form a pusher opening.

In some embodiments, a method of forming a tampon applicator includes: providing a cylindrical paper segment having a longitudinal axis, an outer surface and an inner surface surrounding the longitudinal axis, wherein the leading edge portion or the trailing edge portion forms a portion of the outer surface comprising a seam; inserting a first mandrel into a portion of the cylindrical paper segment; engaging a first end region with the first mandrel; and inverting a portion of the first end region of the cylindrical paper segment such that a portion of the first end region is adjacent to an inner surface of the cylindrical paper segment, wherein the edge of the first end region forms a pusher opening.

In some embodiments, a method of forming a tampon applicator includes: providing a cylindrical paper segment having a longitudinal axis, an outer surface and an inner surface surrounding the longitudinal axis, wherein the leading edge portion or the trailing edge portion forms a portion of the outer surface comprising a seam; inserting a first mandrel into the cylindrical paper segment along the longitudinal axis; and inverting a first end region of the cylindrical paper segment such that a portion of a wall of the first end region is adjacent to an inner surface of the cylindrical paper segment.

### BRIEF DESCRIPTION OF THE DRAWINGS

FIG. 1A is a front view of a conventional plastic applicator including a barrel region and a pusher.

FIG. 1B is a front view of a conventional cardboard applicator including a barrel region and a pusher.

FIG. 1C is a front view of a conventional cardboard applicator.

FIG. 2A is a front view of a tampon applicator including a barrel region and a pusher.

FIG. 2B is a cross sectional view of the tampon applicator of FIG. 2A taken along the line 2B-2B.

FIG. 2C is a side view of a pusher of the tampon applicator of FIG. 2A with a rolled edge at a second end region thereof.

FIG. 2D is a perspective view of a pusher of the tampon applicator with a rolled edge at a first end region and a flange at a second end region thereof.

FIG. 3A is a cross-sectional side view of a plurality of material layers.

FIG. 3B is a side view of a tampon applicator including a non-linear seam.

FIG. 3C is a side view of a tampon applicator including a linear seam.

FIG. 3D is a cross sectional view of the tampon applicator of FIG. 3B taken along lines 3D-3D.

FIG. 3E is a cross sectional view of the tampon applicator of FIG. 3C taken along lines 3E-3E.

FIG. 4A is a side view of an outer member.

FIG. 4B is a perspective view of an outer member.

FIG. 4C is a perspective view of the cross section of a portion of grip region of a tampon applicator.

FIG. 4D is an end view of a tampon applicator including a grip region.

FIG. 5 is a cross sectional view of the grip region of a tampon applicator and a portion of the pusher extending through a pusher opening.

FIG. 6 is a perspective view of a tampon applicator including a grip region.

FIG. 7A is a side view of a portion of the outer member including petals and indicium.

FIG. 7B is a side view of a portion of a first and a second outer member including petals and indicium.

FIG. 8A is a top view of one or more layers of material including a coating and a winding apparatus.

FIG. 8B is a top view of one or more layers of material including a coating and a winding apparatus.

FIG. 8C is a cross sectional view of the one or more layers of material including a coating of FIG. 8B taken along line 8C-8C.

FIG. 9A is a top view of one or more layers of material including a coating and a winding apparatus.

FIG. 9B is a cross sectional view of the one or more layers of material including a coating of FIG. 9A taken along line 9B-9B.

FIG. 10A is a perspective view of a manufacturing process for winding one or more layers of material to form a cylindrical paper segment.

FIG. 10B is a perspective view of a discrete segment.

FIG. 10C is a side view of a discrete segment including a first layer and a second layer including a cut-out portion.

FIG. 10D is a side view of a discrete segment including a first layer and a second layer including a cut-out portion.

FIG. 10E is a perspective view of cylindrical segment including a seam.

FIG. 11A is a side view of a pleating mandrel.

FIG. 11B is a perspective view of a pleating die.

FIG. 11C is a side view of a pleating mandrel operative engaged with a pleating die.

FIG. 11D is a perspective view of a pleated cylindrical segment.

FIG. 12A is a perspective view of a compression device, a first mandrel, a second mandrel, and a forming rod operatively engaging a cylindrical segment to form a grip region.

FIG. 12B is a side view of a compression device, a first mandrel, a second mandrel, and a forming rod operatively engaging a cylindrical segment to form a grip region.

FIG. 12C is a side view of a compression device, a first mandrel, a second mandrel, and a forming rod operatively engaging a cylindrical segment to form a grip region.

FIG. 12D is a side view of a compression device, a first mandrel, and a second mandrel operatively engaging a cylindrical segment to form a grip region.

FIG. 12E is a side view of a compression device, a first mandrel, and a second mandrel operatively engaging a cylindrical segment to form a grip region.

FIG. 12F is a perspective view of an outer member including a grip region having an inverted region and a fold.

FIG. 13A is a perspective view of a cylindrical segment operative engaged with a dome forming mandrel and a dome forming die to from a domed cylindrical segment.

FIG. 13B is a cross sectional view of a cylindrical segment operative engaged with a dome forming mandrel and a dome forming die to from a domed cylindrical segment.

FIG. 13C is a perspective view of a domed cylindrical segment.

FIG. 13D is a cross sectional view of a domed cylindrical segment.

FIG. 14A is a cross sectional side view of a first mandrel and a second mandrel operatively engaging a domed cylindrical segment.

FIG. 14B is a cross sectional side view of a first mandrel and a second mandrel inverting a domed cylindrical segment.

FIG. 14C is a cross sectional side view of a first mandrel and a second mandrel inverting a domed cylindrical segment and a compression device engaging the inverted cylindrical segment.

FIG. 15A is a front view of a compression device including four chucks in a closed configuration.

FIG. 15B is a front view of a compression device including four chucks in an open configuration.

FIG. 15C is a front view of a compression device including eight chucks in an open configuration.

FIG. 15D is a front view of a compression device including eight chucks such that four chucks are in a closed configuration and four chucks are in an open configuration.

FIG. 15E is a front view of a compression device including eight chucks in a closed configuration.

FIG. 16A is a side view of a tampon applicator including a grip region including ridges and an indicium.

FIG. 16B is a perspective view of a tampon applicator including a grip region including ridges and an indicium.

### DETAILED DESCRIPTION

The following term explanations may be useful in understanding the present disclosure.

As used herein the term “tampon” refers to any type of absorbent structure which is configured to be inserted into the vaginal cavity for the interception and absorption of fluid therefrom. Typically, a tampon includes a pledget structure including a quantity of absorbent material, often absorbent fibrous material, which pledget structure has been bunched, folded and/or compressed in one or more lateral/radial directions, the longitudinal direction, or both, via application of pressure, heat and/or moisture control, in order to provide a formed tampon having a size, shape (typically approximately cylindrical) and stability of form to facilitate insertion into the vagina. A tampon which has been so formed is referred to herein has a “self-sustaining” form. The degree of compression, heat and moisture control applied to the pledget is sufficient such that in the subsequent absence of the external forces and absence of substantial contact with moisture, the pledget will tend to retain its general formed shape and size. It will be understood by persons of ordinary skill in the art that this self-sustaining form typically does not persist following insertion of the tampon. Once the tampon is inserted and begins to contact and absorb fluid, the pledget will swell with absorbed fluid and lose its self-sustaining form.

As used herein the terms “pledget” or “tampon pledget” are intended to be interchangeable and refer to a structure including absorbent material configured to perform the primary function of the tampon, absorption of menstrual fluid. A tampon pledget is sometimes referred to as a tampon blank, or a softwind, and the term “pledget” is intended to include structures designated by such terms as well.

As used herein “vaginal cavity” refers to the internal space within the genitalia of the human female, located between the introitus of the vagina (sometimes referred to as the sphincter of the vagina) and the cervix.

With respect to a tampon and an applicator, the “longitudinal” direction is the ordinary general direction of ejection from an applicator; and also corresponds with the ordinary general direction of insertion of a tampon and applicator into, and their withdrawal from, the vaginal cavity in normal use. For a completely manufactured, pre-use tampon that has a pledget with a generally cylindrical or capsule-shaped self-sustaining form, the longitudinal axis of the form lies generally or approximately along the longitudinal direction. A “radial” or “circumferential” direction is a direction perpendicular to the longitudinal direction. The “circumferential” direction is perpendicular to the longitudinal direction and the radial direction, and perpendicular to the z direction (defined below). Unless otherwise specified, references to “length” herein refer to a dimension along the longitudinal direction; references to “width” herein refer to a dimension along the circumferential and/or radial direction.

With respect to a tampon and an applicator, the term “forward” refers to a longitudinal direction of movement during normal insertion of the tampon and/or applicator by a user, and refers to portions of the tampon or applicator that lie closer to and/or enter the vaginal cavity earlier than other portions during normal insertion. Conversely, the term “rearward” refers to a longitudinal direction of movement during normal withdrawal of the tampon and/or applicator by a user, and refers to portions of the tampon or applicator that lie farther from and/or exit the vaginal cavity earlier than other portions during normal withdrawal.

A “nonwoven,” “nonwoven web,” “nonwoven web material,” or “nonwoven fabric” is a cloth-like web material (or portion or section thereof) formed predominantly of fibers that are neither knitted nor woven, but rather, laid down and accumulated to a desired basis weight, then consolidated and held together to form a web, via one or any combination of calendering, thermal and/or compression bonding, bonding via use of a binder, heating (via, e.g., heated air driven through an accumulation of fibers) or hydroentangling (spunlace). The predominant fibers may be natural fibers harvested from plant material (e.g., cotton) (but excluding tree wood pulp), semi-synthetic (e.g., rayon, lyocell, viscose), or synthetic (e.g., fibers spun from molten thermoplastic polymer resin(s)), or any combination thereof. Herein, a skin-or membrane-like film (e.g., extruded or otherwise formed from polymer resin(s)) is not deemed a nonwoven. Herein, a paper tissue product, paper product, or paperboard or cardboard product, formed via wetlaying and predominantly constituted of tree wood pulp, is not deemed a nonwoven.

“Opened configuration,” with respect to a tampon, means the configuration of the pledget prior to the time it is compressed and formed into a self-sustaining form during manufacture, or in the case of a finished product, after it is completely ejected from an applicator (if present) and/or allowed and/or caused by any suitable technique to open and substantially re-assume its pre-compression shape and size.

“Paper” or “Cardboard” means a material in web or sheet form, formed predominantly of cellulose fibers, for example, wood pulp fibers, which have been suspended in a slurry, which is then poured onto a moving mesh belt, drained of water, and subsequently dried over drying rollers, and in many examples, finished via calendaring, or which is injection molded and subsequently dried, or which is formed by pulp molding. In the resulting web or sheet product the cellulose fibers are interlaid and randomly oriented. However, due to the direction of manufacture, the majority of fibers may be oriented in the machine direction. Thus, the fibers may be semi-randomly oriented. The paper or cardboard herein may also be substantially void of synthetic materials as defined herein, such as fibers, films, and/or adhesives.

“Predominant,” and forms thereof, when used to characterize a quantity of a constituent present in a composition, means that a majority of the weight of the material is constituted by the constituent.

“Withdrawal cord” refers to any section of string, yarn, cord, ribbon, strip material or other flexible/pliable elongate structure typically (although not necessarily) formed of fibrous material, attached to and/or extending from a tampon pledget and trailing from its rearward end. A withdrawal cord of sufficient length may be provided with a tampon for the purpose of providing a relatively thin and flexible trailing member of sufficient length to allow for a portion thereof to trail and remain outside of the introitus following full insertion of the tampon, which the user may easily grasp and pull to withdraw the tampon from her body following a desired duration of use.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

With respect to a paper sheet or web product, when laid out flat on a horizontal planar surface, the “z direction” is a direction orthogonal to the horizontal planar surface and is the direction along which caliper or thickness of the sheet or web (prior to rolling into paper tube product) would be measured.

### Applicator Features

The present disclosure is related to absorbent articles including a tampon, and more particularly, to a tampon having an outer member and an inner member such that the inner member is slidably engaged with the outer member. The outer member includes a cylindrical paper segment defining a longitudinal applicator axis. The cylindrical paper segment includes an outer surface and an inner surface that surround the longitudinal axis. The cylindrical paper segment includes an insertion end region, a grip end region opposite the insertion end region, and an intermediate region between the insertion end region and the grip end region. The grip end region of the cylindrical paper segment provides an improved user grip for a cardboard tampon applicator, as will be described herein. This improved grip region provides greater functionality and case of use by providing an enhanced area for the user to grip and securely hold the tampon applicator during use. The cylindrical paper segment may include one or more layers of material, such as paper, that are spirally or convolutely wound to form the cylindrical paper segment. Each of these one or more layers may comprise, consist essentially of, or consist of cellulosic material. Stated another way, each of the one or more layers may be void of synthetic fibers and films. A carboard applicator, as referred to herein, is an applicator that includes one or more layers and each of the one or more layers comprise, consists essentially of, or consists of cellulosic material and is void of synthetic fibers and films. “Synthetic” means that the polymerization did not happen in nature or was not naturally occurring. Synthetic materials are those that are chemically modified such that the polymerization was not naturally occurring. Synthetic fibers, for example, include synthetic polymeric materials and bio-plastic polymers, such as polyethylene (PE), polyethylene terephthalate (PET), polylactic acid (PLA), polyhydroxyalkanoates, polybutylene succinate. Materials that are not synthetic, for example, include cotton, flax, hemp, rayon, and jute. Void of synthetic materials or free of synthetic materials means that the article or component of the article is at least about 90% or at least 95% or at least about 98% by weight void of materials where the polymerization did not happen in nature or was not naturally occurring.

Thus, the tampon of the present disclosure may have a cardboard applicator that provides greater functionality and convenience, and a more appealing ergonomic and/or aesthetic experience.

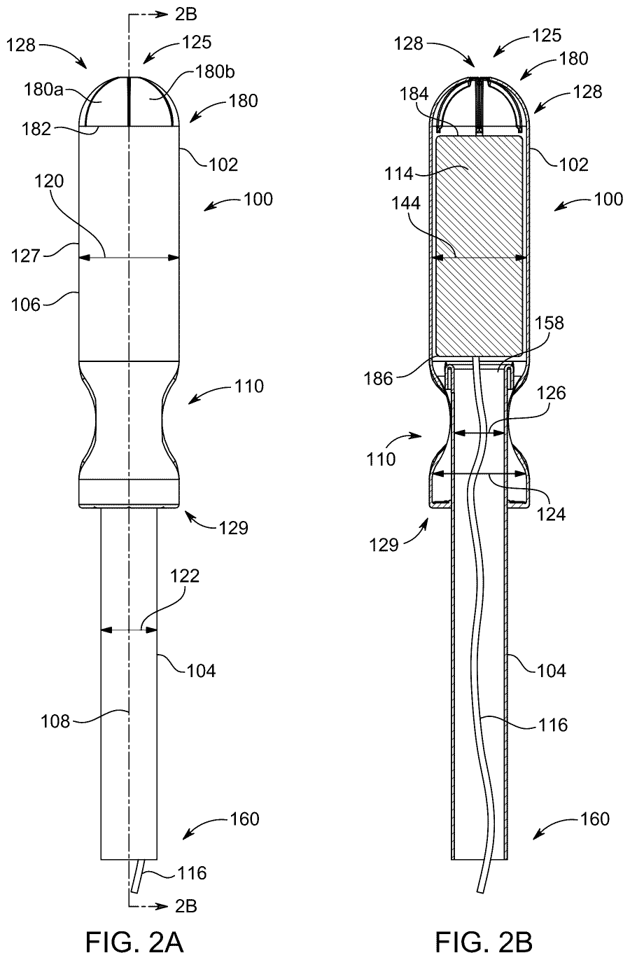

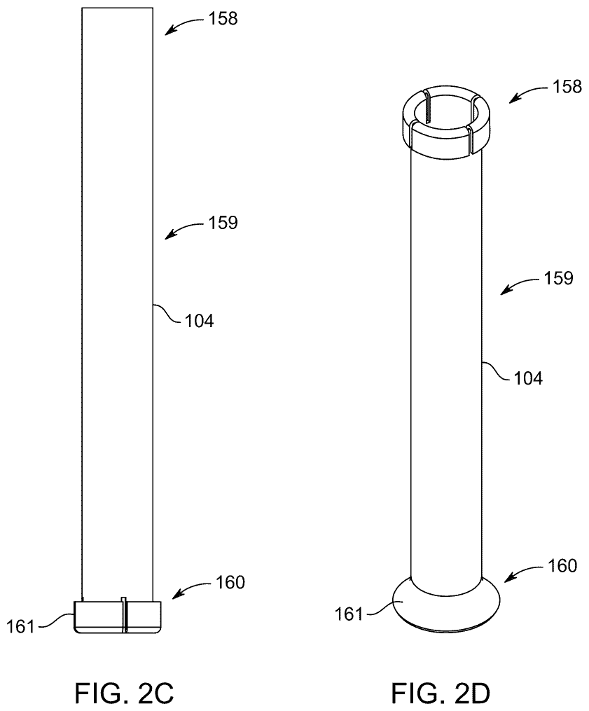

Referring to FIGS. 2A and 2B, a tampon in a self-sustaining form, including a pledget 114 having a forward end 184 and rearward end 186, and a withdrawal cord 116 attached or joined to the pledget and trailing from the rearward end 186, may be supplied inside an applicator 100. Applicator 100 may be configured to house the tampon pledget and protect it from contamination and/or unintended deformation prior to its use.

Applicator 100 may include an outer member including a hollow outer member 102 and an inner member 104 (also referred to herein as an ejection pusher or pusher), which also may be hollow. The outer member 102 and the inner member 104 may be configured such that the inner member 104 is at least partially disposed and longitudinally slidable within the outer member 102. As illustrated in FIGS. 2A and 2B, each of the outer member 102 and the inner member 104 are hollow approximately cylindrical bodies comprising a cylindrical paper segment. The cylindrical paper segment of the outer member 102 includes an insertion end region 128, a grip end region or grip region 110 opposite the insertion end region 128, and an intermediate region 127. The inner member 104 has an external diameter 122 that is smaller than an inner diameter 144 of the intermediate region 127 of the outer member 102, such that the outer member 102 and the inner member104 may be concentrically arranged and longitudinally slidable with respect to each other in a telescoping manner. In some embodiments, the external diameter of the of the intermediate region of the pusher or inner member is less than about 70% of the external diameter of the intermediate region of the outer member.

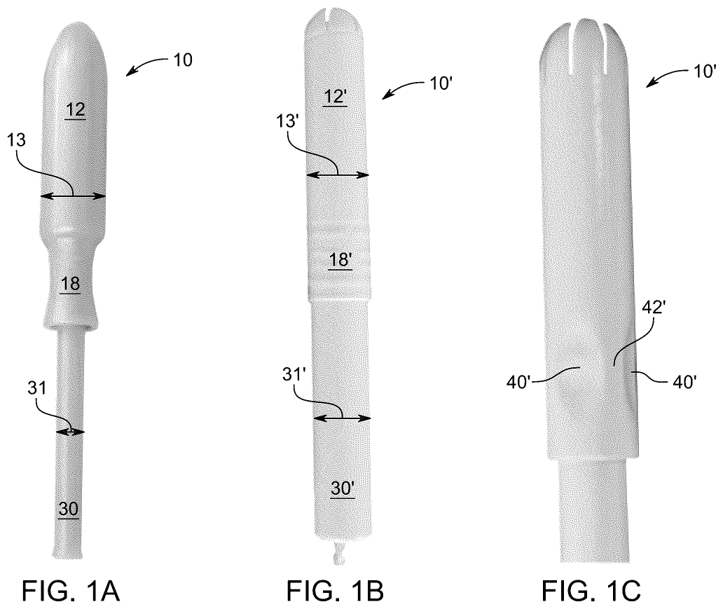

As shown in FIG. 2A, the outer member includes a cylindrical paper segment defining a longitudinal axis and having an outer surface 106 surrounding the longitudinal applicator axis 108. The cylindrical paper segment of the outer member 102 includes an insertion end region 128, a grip end region or grip region 110 opposite the insertion end region 128, and an intermediate region 127. The outer member 102 has an external diameter 120 that is greater than the external diameter 122 of the pusher 104. The external diameter 120 of the barrel region is about 16 mm or in a range from about 8 mm to about 22 mm or from about 12 mm to about 20 mm or from about 12 mm to about 18 mm, and the external diameter 122 of the inner member is about 7 mm or in a range from about 2 mm to about 18 mm or from about 4 mm to about 12 mm or from about 6 mm to about 9 mm. The external diameter 122 of the intermediate region of the pusher 104 may be less than about 70% of the external diameter 120 of the intermediate region of the outer member 102. Unlike the conventional cardboard applicator 10′ (FIG. 1B) where the external diameter 31′ of the pusher 30′ is about 80-90% of the external diameter 13′ of the barrel region 12′, the external diameter 122 of the pusher 104 is about 50%-60% of the external diameter 120 of the intermediate region of the outer member 102. The inventors of the present disclosure recognized that the external diameter 31′ of the pusher 30′ in the conventional cardboard applicator 10′ had to be at least 80-90% of the external diameter 13′ of the barrel region 12′ to avoid the pusher 30′ wobbling as it is slid into or withdrawn from the barrel region 12′. The improved cardboard applicator 100 herein discloses an improved grip region 110 to accommodate the external diameter 122 of the pusher 104 only being 50-60% of the external diameter 120 of the outer member 102 in order to prevent the undesired wobbling between the inner member 104 and the outer member 102.

The outer member 102 may be provided with additional functional features. For example, a forward end 125 of the outer member may include an insertion end region 128 adjacent to the intermediate region 127. The insertion end region 128 includes at least two petals 180 that converge toward the longitudinal axis 108. The at least two petals have proximal bases 180a having outer surfaces coincident with the general cylindrical shape of the outer member 102, and distal ends 180b disposed radially inwardly of the proximal portions. The insertion region 128 of the outer member 102 is opposite from the grip region 110. The petals 180 and/or remainder of the intermediate region 127 may be configured such that the petals 180 may flex about the proximal bases 180a such that, upon forward longitudinal pressure exerted by the forward end 184 of the pledget 114 during ejection via pressure form longitudinal forward movement of the ejection pusher 104 relative to the outer member 102, the petals 180 will flex outwardly about their bases. This expands an opening at the insertion end region 128, which allows the pledget 114 to pass forwardly through the forward end 125 of the tampon applicator or the insertion region 128 of the outer member 102, and out of the applicator 100. The petals 180 may be integrally formed with the remainder of the outer member 102. In some examples, the petals may be formed by making cuts in a forward portion of a cylindrical paper segment of the outer member 102, and then plastically deforming the remaining uncut portions radially inwardly, and with suitable curvature. Such deformation may be accomplished in some examples via use of a forming die or mold (not shown), and may be facilitated in some examples via application of heat and/or steam and/or added moisture, such as the addition of water or humidification of the environment in which the applicator or a portion thereof is formed. The outer member 102 may be imparted with localized flexure zones 182 that demark the proximal bases 180a of the petals, which are zones about which the material of the cylindrical paper segment and/or petals more readily flexes or hinges, for example, as a result of a localized reduction in thickness or caliper or weakening of the material in flexure zones 182. Flexure zones 182 may be imparted by, e.g., molding or localized compression applied during the manufacturing process. These localized flexure zones may be formed at the bases of the petals and/or throughout other regions of the petals to aid in the curved or bent formation of the petals in a pre-dispending configuration and to aid in allowing other portions of the petals to bend/flex more easily during dispensing of the tampon pledget.

The outer member 102 including a cylindrical paper segment may also be provided with a grip region 110 having gripping features that enable the user to tactilely identify a rearward end region 129 of the outer member 102, and also enhance gripping slip resistance and ergonomics during use of the applicator 100.

As illustrated in FIG. 2B, the outer member 102 may be sized to house the tampon pledget 114 in its self-sustaining form. It may be desired that the outer member 102 have an internal diameter 144 that is small enough relative the outer lateral dimension of the pledget 114 such that the pledget 114 cannot freely slide longitudinally within the barrel region 102 (due to slight interference or radial compression and light static friction resistance), but not so small as to create unacceptable static friction resistance and thereby require the user to exert unacceptably great force on the ejection pusher or inner member 104 to eject the pledget 114 from the barrel region 102, when ejection is desired. It is to be appreciated that the internal diameter of the outer member may be such that the pledget 114 moves freely within at least a portion of the outer member. The pledget 114 may be disposed within at least a portion of an intermediate region 127 of the cylindrical paper segment of the outer member.

Also as suggested in FIG. 2A, it may be desired that the ejection pusher 104 have at least a portion having an external diameter 122 (of its cylindrical form) that is equal to or smaller than the internal diameter 144 or other inner lateral dimension of the outer member 102. It may be desired to provide such clearance within 1% of parity such that the ejection pusher 104 cannot freely and unintentionally slide coaxially/longitudinally within or out of the outer member 102 in the absence of intentionally applied force, but such that the force required to slide the ejection pusher 104 within the outer member 102 is not so great as to be unacceptably great to the user, or even potentially destructive to the applicator. It is to be appreciated that the first end region and the second end region of the pusher may have an external diameter that is greater than the external diameter of the region of the pusher that extends through the pusher opening in the outer member, which prevents the pusher from inadvertently being removed from the outer member in a pre-dispensing configuration (to prevent the pusher from disengaging the outer member) and a post-dispensing configuration (to prevent the pusher from being expelled with the pledget).

Referring to FIGS. 2A and 2B, the ejection pusher 104 may be provided with an inner diameter that is smaller than an outer diameter or lateral dimension of the tampon pledget 114 (or other object) housed within the outer member 102. With this size relationship, longitudinal force applied to the ejection pusher 104 urging it into the outer member 102 will cause its forward end or first end region 158 to contact the rearward end 186 of the tampon pledget 114 (or other object) and urge the pledget forward and out the forward end 128 of the outer member 102.

As an alternative, or in addition to, the forward end region or first end region 158 of the ejection pusher 104 may have its circumferential edge rolled over inwardly or otherwise formed to extend radially inwardly, thereby effectively reducing the inner diameter or inner lateral dimension of the ejection pusher 104 at its forward end 158.

The first end region 158 of the ejection pusher or inner member 104 may be outwardly rolled or flanged to increase the amount of area at the forward end 158 of the ejection pusher 104 that effectively contacts the rearward end 186 of the pledget 114 or other object within the outer member 102, to transmit ejection force exerted by the user. A similar feature can be imparted to the rearward end or second end region 160 of the ejection pusher 104, to expand contact surface area and decrease focused/localized pressure on the user's finger at the circumferential rim of the rearward end 160 when the user applies ejection force at the rearward end 160, and thereby make application of ejection force more comfortable to the user's finger. The pledget 114 may be configured with an expulsion force of less than 900 gram-force (gf) or less than 800 gram-force (gf) or less than about 650 gf or in a range of from about 400 gf to about 450 gf or from about 400 gf to about 650 gf or from about 250 gf and about 700 gf or from about 200 gf to about 1200 gf. Generally, consumers prefer a pusher with a first external diameter in an intermediate region, a second external diameter in the forward end, and a third external diameter in the reward end, and each of the second external diameter and the third external diameter are greater than the first external diameter. Again, having regions of the pusher that have greater external diameters in the forward end region and the reward end region allow for the pusher to remain slidably engaged with the outer member both pre and post dispending and to provide a more ergonomic, greater surface area on which the consumer can interact while using the tampon applicator.

In some embodiments, the outer member 102 is manufactured such that the internal diameter 144 thereof is less than about 19 mm and preferably less than about 18 mm or from about 9 mm to about 18 mm or from about 11 mm to about 17 mm. Although the external diameter of typical menstrual-use tampons varies, most menstrual-use tampons currently marketed have an external diameter of less than about 19 mm. However, if one desires to use the applicator of the present disclosure for purposes other than delivery of a menstrual-use tampon to a human vaginal cavity (such as, for example, delivery of other devices, medications, etc. to other cavities in humans, or cavities in animals (veterinary use)), an outer member 102 with a larger diameter may be desired.

The material of the outer member may be overlapped into a tubular configuration forming a cylindrical paper segment defining the longitudinal axis. The cylindrical paper segment of the outer member may be formed by spirally or convolutely winding one or more layers of material. A seam is formed in the cylindrical paper segment. Depending on the type of winding, convolute or spiral, the seam may be substantially parallel to the longitudinal axis or the entire seam or a portion of the seam may not be substantially parallel to the longitudinal axis. Further, the edge of the one or more layers may be non-linear, which also may result in a scam or a portion thereof not being substantially parallel to the longitudinal axis. The cylindrical paper segment may be formed from a single layer of material, such as a single sheet of paper, or multiple layers of material. The one or more layers forming the cylindrical paper segment may be wound about the longitudinal axis or a winding axis one or more revolutions or from about 1 or more revolutions or about 2 or more revolutions, or from about 1.25 revolutions to about 20 revolutions.

The inner member 104 may be manufactured of similar materials and in a similar layered and wound configurations as the outer member, with appropriate adjustments to its size (length and outer diameter) to enable it to fit within the outer member 102 with appropriate clearance, and have sufficient length to facilitate full ejection of the tampon pledget (or other object) from the outer member 102.

For a menstrual-use tampon, the outer member 102 may be sized and configured to snugly house the tampon pledget. As noted above, it may be desired that the outer member have a relatively smooth exterior surface that will facilitate comfortable insertion of the outer member 102 into the vaginal cavity. When the exterior surface is smooth (and preferably has surface properties and/or surface treatment that provide appropriately low friction with sensitive tissue), the outer member 102 will easily slide within the vaginal cavity without subjecting the internal tissues of the vagina to irritation or abrasion. The exterior surface of the outer member 102 may be coated to give it appropriate low friction characteristic surface properties. Wax, polyethylene, a combination of wax and polyethylene, acrylic, polybutylene succinate, and cellophane are representative coatings or components of coatings that may be applied to the outer member of tampon applicators, to facilitate comfortable use.

The applicator of the present disclosure may be used for the delivery of menstrual-use devices, such as a tampon pledget, an intravaginal collection device (e.g., menstrual cup), and interlabial pads. The applicator of the present disclosure may be used for the delivery of a pessary. The applicator may also be useful for delivery of oral, rectal, and vaginal suppositories, as well as nasal devices, such as nasal tampons. The applicator may be used for delivery of various other materials including, medicaments, moisturizers, vitamins and minerals, spermicides, and odor controlling agents. These materials may be delivered in the form of rigid solid objects, deformable solid objects, creams, foams, gels, etc. The applicator may be adapted for human or animal/veterinary use.

FIG. 2B depicts a non-limiting example of a tampon having the pledget 114 in a self-sustaining form and having a forward end 184 and a rearward end 186 and a withdrawal cord 116 attached to the pledget 114 and extending rearward from a location proximate the rearward end 186 of the pledget 114. Generally, the “pledget” as referred to herein is that portion of the tampon that includes the main body and mass of absorbent material, but does not include the withdrawal cord 116.

Tampons contemplated herein, however, are not limited to structures having the particular configuration shown in the drawing. In some embodiments, at least a portion of the cord 116 is braided. In other embodiments, there may be two different portions of the cord 116 that are each braided. In this example embodiment, one of the braided portions is a thick portion and the other of the braided portions is a thinner portion, as disclosed in U.S. Patent Publication No. 2023/0058874 that is incorporated by reference herein.

As shown in FIG. 2B, the pledget 114 of the tampon has a forward end 184 and a rearward end 186. During manufacture of the tampon pledget, the pledget 114 may be folded, bunched, compressed and/or otherwise formed in size and shape, from its initially manufactured configuration into a generally cylindrical and/or capsule-shaped configuration (e.g., as shown in FIG. 2B) along a radial direction, the lateral direction, longitudinal direction, or in some combination thereof.

FIG. 2C is a longitudinal side view of a pusher 104 with a flange 161 at a second end region 160 thereof. The pusher 104 includes a forward end 158 and a rearward end 160 opposite to the forward end 158. During use of the applicator 100, the forward end 158 of the pusher 104 is configured to engage a portion of the pledget 114 and the rearward end 160 includes the flange 161. In some embodiments, the flange 161 may have a diameter greater than the internal diameter of the barrel region 102 at the rearward end 129 or the flange may have a diameter greater than a portion of the internal diameter of the grip region 110, to keep the pusher 104 from unintentionally going through the barrel region 102 and out the forward end of the barrel, and to provide a surface on which the consumer or user can push to expel the pledget 114 from the forward end 128 of the outer member 102, such as shown on FIG. 2D. The flange 161 may be a pushed-out petal, a compressed portion, an embossed portion, and/or added material to increase the diameter of the pusher 104 at the rearward end 160. It is also to be appreciated that each of the first end region and/or the second end region may include a portion that has been rolled inwardly or outwardly or flanged.

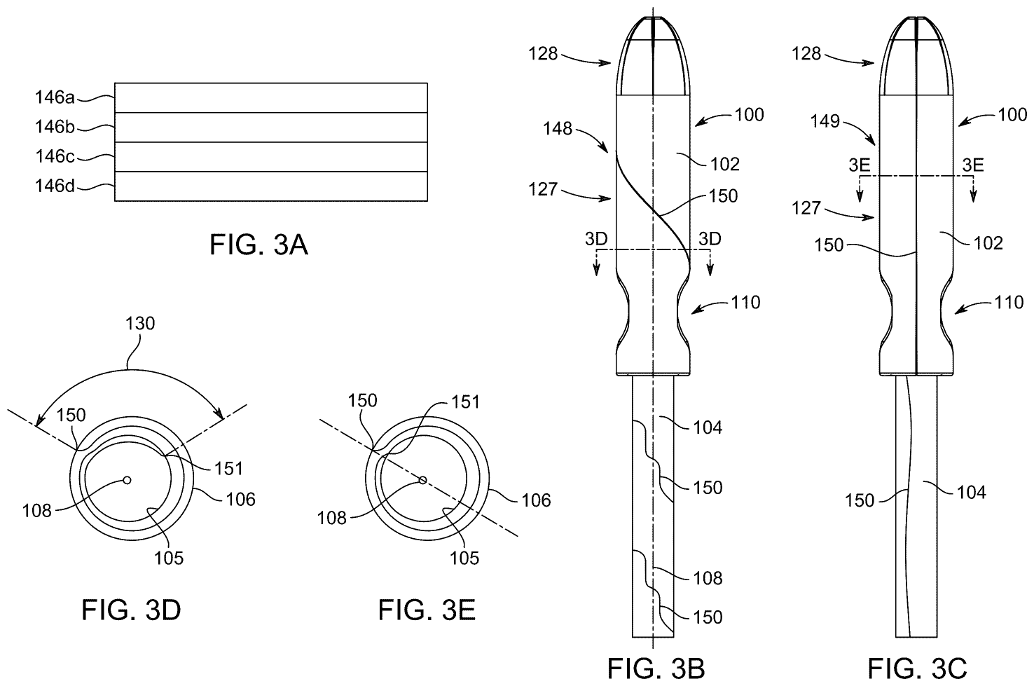

FIG. 3A is a cross-sectional side view of a plurality of material layers 146a through 146d that may be wound to form the cylindrical paper segment of the outer member and the inner member. Although four layers 146a through 146d are illustrated in FIG. 3A, in other embodiments less or more than four layers may form each of the inner member and the outer member. The one or more layers may be positioned in a staggered relationship to one another or with respect to the adjacent layer or with respect to one or more other layers. The one or more layers may be positioned such that one or more edges of the layers are staggered or offset and/or the one or more layers may be positioned such that one or more edges are aligned. Additionally, it is to be appreciated that a single layer of material may be wound to form the cylindrical paper segment of the outer member and/or the inner member. Likewise, multiple layers of material may be wound to form the cylindrical paper segment.

The one or more of the layers 146a through 146d may exclude or be void of non-naturally occurring fibers. The one or more layers 146a through 146d may include naturally-occurring fibers. Naturally occurring fibers may include cellulosic fibers, wool, silk and other naturally-occurring protein fibers and mixtures thereof, cotton, cotton linters, flax, sisal, abaca, hemp, hesperaloe, jute, bamboo, bagasse, kudzu, corn, sorghum, gourd, agave, loofah, trichomes, seed-hairs, wheat, and mixtures thereof. In other embodiments, each of the one or more layers 146a through 146d include cellulosic material including wood fibers. Wood fiber(s) means fibers derived from both deciduous trees (hereinafter, also referred to as “hardwood”) and coniferous trees (hereinafter, also referred to as “softwood”) may be utilized. Wood fibers may be short (typical of hardwood fibers) or long (typical of softwood fibers). Nonlimiting examples of short fibers include fibers derived from a fiber source selected from the group consisting of Acacia, Eucalyptus, Maple, Oak, Aspen, Birch, Cottonwood, Alder, Ash, Cherry, Elm, Hickory, Poplar, Gum, Walnut, Locust, Sycamore, Beech, Catalpa, Sassafras, Gmelina, Albizia, Anthocephalus, and Magnolia. Nonlimiting examples of long fibers include fibers derived from Pine, Spruce, Fir, Tamarack, Hemlock, Cypress, and Cedar. The one or more of the layers 146a through 146d may be free of synthetic fibers and films. In some embodiments, one or more of the layers 146a through 146d may include other materials, such as plastic. However, in other embodiments, each of the layers 146a through 146d are 100% cellulosic. In some embodiments, the cellulosic material of the one or more layers 146a through 146d is paper. In other embodiments, the cellulosic material of the one or more layers 146a through 146d includes pulp.

FIGS. 3B and 3C are each a side view of a tampon applicator 100 including an outer member and an inner member formed from one or more material layers, such as for example the material layers 146a through 146d of FIG. 3A. The one or more layers may be spirally wound or convolutely wound to form the outer member and the inner member. When the one or more layers are wound, an external seam is formed on an outer surface of the outer member and the inner member. A scam is a region of the leading-most and/or trailing-most layer edge that bonds to a subsequent or previous revolution to form the cylindrical segment. A seam is disposed on the outer surface and/or the inner surface of the cylindrical segment which may form an inner member and/or an outer member. FIG. 3B illustrates a spiral or non-linear external seam 150 formed when the one or more material layers are spirally wound to form the outer member. Similarly, the inner member includes a spiral or non-linear external scam 150 form when one or more layers of material are spirally wound. The non-linear scam may not be parallel to the longitudinal axis or at least a portion of the non-linear seam may not be parallel to the longitudinal axis 108. The spiral or non-linear external seam 150 may extend from the insertion region through the intermediate region and into the grip region. The insertion region may or may not have a scam or a portion thereof. For example, the insertion region may include one or more petals that are formed from one or more layers of material that are free to pivot and move allowing expulsion of the pledget, and thus, would be hindered if the external seam extended into the region including the petals. Further or alternatively, the external seam may not be needed in the insertion region for the outer member to maintain its cylindrical shape. The one or more layers may be configured such that no external seam is formed in the insertion region when the one or more layers are wound due to the shape of the layer. For example, the layer may be cut or folded such that when the layer is wound an external seam 150 is not present in the insertion region. The portion of the external seam extending into the grip region may or may not be visually discernable due to the formation of the grip region. A visually discernible difference exists if an observer in ordinary indoor lighting conditions (20/20 vision, lighting sufficient to read by, for example) can visually discern a difference between the revolutions from the outer surface for an external scam and the inner surface for an internal seam.

FIG. 3C illustrates a linear external seam 150 formed when the one or more material layers are convolutely wound to form the outer member. Similarly, the inner member includes a linear external seam 150 form when one or more layers of material are convolutely wound. The linear external seam may be substantially parallel to the longitudinal axis 108. The linear external scam 150 may extend from the insertion region through the intermediate region and into the grip region. The insertion region may or may not have a scam or a portion thereof. For example, the insertion region may include one or more petals that are formed from one or more layers of material that are free to pivot and move allowing expulsion of the pledget, and thus, would be hindered if the external scam extended into the region including the petals. Further or alternatively, the external seam may not be needed in the insertion region for the outer member to maintain its cylindrical shape. The one or more layers may be configured such that no external seam is formed in the insertion region when the one or more layers are wound due to the shape of the layer. For example, the layer may be cut or folded such that when the layer is wound an external seam 150 is not present in the insertion region. The portion of the external seam extending into the grip region may or may not be visually discernable due to the formation of the grip region.

FIG. 3D illustrates a cross-sectional view of the tampon applicator taken along lines 3D-3D of FIG. 3C. The tampon application may be wound about a winding axis, which may be substantially parallel to the longitudinal axis 108. The one or more layers make one or more revolutions about the longitudinal axis 108. The one or more layers may make one revolution or from about one revolution to about 20 revolutions or from about 2 revolutions to about 10 revolutions or from about 2 revolutions to about 8 revolutions or from about 3 revolutions to about 6 revolutions or from about 3 revolutions to about 4 revolutions. It is also to be appreciated that after the full first revolution, a partial revolution may be made on any subsequent revolution. It is also to be appreciated that depending on the placement of two or more layers with respect to one another, not every layer will make the same number of revolutions. For example, a second layer that is substantially shorter than an outer, first layer may make less revolutions that the relatively longer outer, first layer. An internal scam 151 is formed by an edge of the one or more layers and is disposed on an internal or inner surface of the tampon applicator. The internal seam 151 or at least a portion thereof may be substantially parallel to the longitudinal axis. The internal seam 151 or at least a portion thereof may not be substantially parallel to the longitudinal axis.

With reference to FIG. 3D, the internal seam 151 may be separated from the external seam 150 by a scam separation distance 130 measured circumferentially about the outer surface of the cylindrical paper segment of the member. The seam separation distance 130 may be less than about 8 mm or less than about 5 mm or less than about 3 mm or less than about 2 mm or from 0 mm to about 8 mm or from about 1 mm to about 5 mm or from about 2 mm to about 3 mm. It is to be appreciated that the outer surface may be non-circular and, thus, the seam separation distance is measure along the outer surface from the first seam to the second seam. As illustrated in FIG. 3E, the external scam 150 and the internal seam 151 may be radially aligned. The placement of the internal and external seam with respect to one another may be based on the shape of the edges of the one or more layers. The placement of the internal and external seam with respect to one another may vary along the longitudinal axis.

It is to be appreciated that the placement of the seam and winding of the one or more layers as described above applies to each of the inner member and the outer member.

The outer member may be provided in the form of a spirally wound, convolutely wound or other seamed hollow tube which may be formed from paper, paperboard, cardboard or a combination thereof. The paper, paperboard, or cardboard may be manufactured predominantly of wood and/or cellulose pulp fiber. The outer member 102 may be manufactured to be relatively rigid and have an external barrel diameter 120 of about 10 millimeters to about 20 millimeters. The outer member 102 has a wall which may be manufactured to have a predetermined thickness of about 0.1 mm to about 0.7 mm. The wall may be constructed from a single layer of material or be formed from two or more layers that may be joined together.

The use of two or more layers enables the manufacturer to use particularly selected materials in the various layers that may enhance the performance of the tampon applicator 100. When two or more layers are included, all the layers may be spirally wound, convolutely wound, and/or longitudinally seamed to form a cylindrical paper segment. It is to be appreciated that a cylindrical segment may be formed by several different winding methods, such as both spirally and convolutely, when multiple layers are used to form the cylindrical segment. For example, a first layer may be spirally wound to form a preliminary cylindrical segment and a second layer may be convolutely wound about the preliminary cylindrical segment to form the cylindrical segment of the outer member and/or inner member. The outer surface may be constructed using a relatively smooth layer of material on the outside or exterior surface that surrounds a relatively coarser and possibly thicker layer. When the wall includes at least three layers, the middle layer may be the thicker layer and the interior and exterior layers may be imparted with a relatively smooth and/or slippery surface finish (e.g., via application of a coating or calendering) to facilitate expulsion of the tampon pledget and to facilitate comfortable insertion of the outer member 102 into the vaginal cavity, respectively. By sandwiching a thick, coarser layer of material between two thin, smooth layers, an outer member 102 may be both economical and functional. The outer member and/or inner member may include one to three, four or five layers, although more layers may be utilized if desired. It is also to be appreciated that the surface of the applicator may be formed to be relatively smooth by a calendering process.

The layers forming the cylindrical segment may be held together by a coating, such as an adhesive, or by a coating activated by heat and/or pressure, or by mechanical interaction such as by ultrasonic bonding or pressure bonding, or a combination thereof. It is also to be appreciated that the coating may be water and the one or more layers may be held together by water induced hydrogen bonding. The coating may be a petroleum, a petroleum derivative, and/or a plastic with petroleum or petroleum derived components or precursor materials. Thus, an applicator may include less than 10 percent by weight or less than 5 percent by weight petroleum, petroleum derivatives and/or plastics with petroleum or petroleum-derived components or precursor materials. For example, each of the layers of the applicator may include a cellulosic material, such as paper, and the coating between the layers may be a petroleum, a petroleum derivative, and/or a plastic with petroleum or petroleum derived components or precursor materials. Alternatively, the applicator may include a coating, such as an adhesive that is not a petroleum, a petroleum derivative, and/or a plastic with petroleum or petroleum derived components or precursor materials. Suitable non-plastic adhesives may also be used.

The type as well as amount of adhesive utilized to bond the outer member and/or inner member of the present disclosure can impact the recyclability of the package. As an example, adhesives which can dissolve in water during the re-pulping stage of the disintegration step of the recycling process may be particularly suitable for the inner/outer member of the present disclosure. Such adhesives include starch based adhesives, polyvinyl alcohol based adhesives, and polyethylene oxide based adhesives. One suitable example of a starch based adhesive is available from LD Davis located in Monroe, N.C., under the trade name AP0420CR. One suitable example of a polyvinyl alcohol based adhesive is available from Sekisui Chemical Company, located in Osaka, Japan, under the trade name Selvol 205. One suitable example of a polyethylene oxide based adhesive is available from Dow Chemicals Co. located in Midland, Mich., under the trade name WSR N-80. If the adhesive is not water-soluble, then water-dispersible adhesives may similarly be utilized. Suitable examples of water dispersible adhesives include thermoplastic elastomer based adhesives and polyvinyl acetate based adhesives. One suitable example of a thermoplastic elastomer based adhesive is available from Actega located in Blue Ash, Ohio, under the trade name Yunico 491. One suitable example of a polyvinyl acetate based adhesive is available from Bostik located in Milwaukee, Wis., under the trade name Aquagrip 4419U01. Another suitable example of a polyvinyl acetate based adhesive is available from HB Fuller under the trade name PD-0330. Any suitable pressure sensitive adhesives may be utilized as well. One suitable example of a pressure sensitive adhesives includes sold by Formulated Polymer Products Ltd. Located in Bury, Lancashire, England, and sold under the trade name FP2154. As one specific example, the access seal may comprise a pressure sensitive adhesive.

The coating may be water-soluble or water-insoluble. For example, a water-soluble coating, such as a water-soluble adhesive, may be preferred, for reasons of environment-related concerns, so that the wound layers will readily separate when wetted by water or relatively prolonged moisture. Such wetting/moisturizing may occur, for example, upon immersion by flushing in a toilet, or upon exposure to environmental moisture (for example, in an outside landfill in moist or wet conditions). When a water-soluble coating is used, exposure of the tampon applicator to processes of a municipal waste treatment plant, wherein soaking in water, interaction with chemicals and agitation all occur, will cause the wound one or more layers to separate and disperse in a relatively short period of time.

In addition to the objective of providing for dissolution and dispersibility, as noted above, it may be desirable to reduce or eliminate components derived from petroleum according to currently recognized objectives relating to use of sustainably-sourced and environmentally-friendly materials. Many (if not all) adhesives currently used to adhere layers of paper together to form tube structures for applicator components are aqueous formulations including polyvinyl acetate (PVAc). PVAc-based adhesive (exemplified by “white glue” or ELMER'S brand glue) is desirable because it is water soluble, readily penetrates pores of fibrous cellulose/wood pulp paper and adheres to cellulose fibers, is tacky when applied (promoting rapid setting and bonding), and can be formulated with relatively high water content and low viscosity for case of application during manufacturing. However, PVAc is derived from hydrocarbons (acetylene or ethylene), most economically obtained from petroleum. Other non-petroleum-derived adhesive components that may be used, include hide glue and sodium silicate (water glass), which are used elsewhere in paper-related applications.

When tube stock used to form an applicator or ejection pusher is formed of paper (in turn, formed of cellulose pulp such as wood pulp), the paper may be relatively hydrophilic, porous and absorbent, and also, potentially abrasive to sensitive tissue prior to a coating being applied. The hydrophilic, porous and absorbent qualities may cause the paper material to draw moisture from moist tissues, and thereby increase the potential for irritation and abrasion from sliding contact therebetween. Additionally, these qualities may increase the potential for staining of the paper material with menstrual fluid, which, following withdrawal of an applicator from the vaginal cavity, some users may find undesirable. Accordingly, it may be desirable to apply a coating to the material that, when in melt and/or liquid form readily penetrates the fibrous structure of the paper and seals it to prevent it from absorbing fluid. The coating may be applied to one or more layers, such as the outer layer or outer surface of the outer member or inner member, and/or the inner layer of the outer member or the inner member. The coating may be applied to the layer prior to forming the applicator. For example, the coating may be applied prior to the one or more layers being convolutely wound and/or spirally wound. Alternatively, or in addition to, a coating may be applied to the outer member and/or the inner member after winding. The coating may be applied in a first state that is uncured and may later be cured. For example, a coating may be applied and may be uncured when the first end region is compressed to form the grip region. After or during formation of the grip region, the coating may be cured, such as be heat, light (such as UV light), or similar methods.

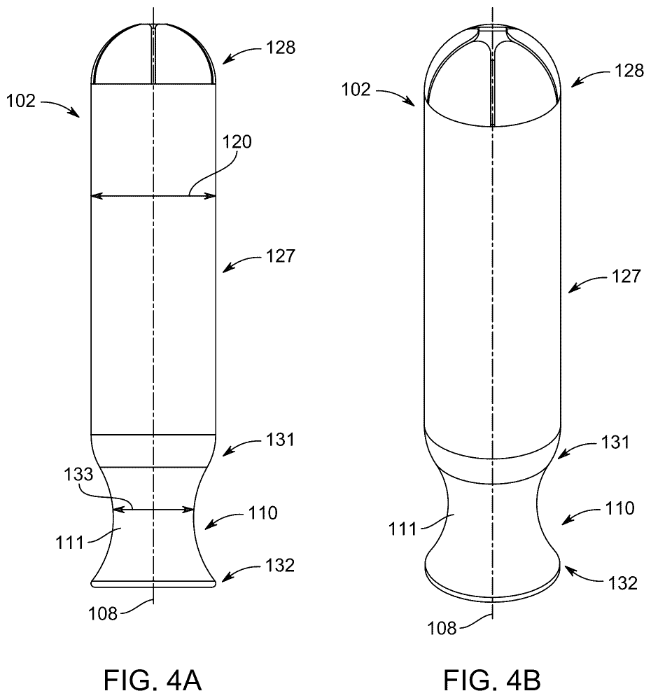

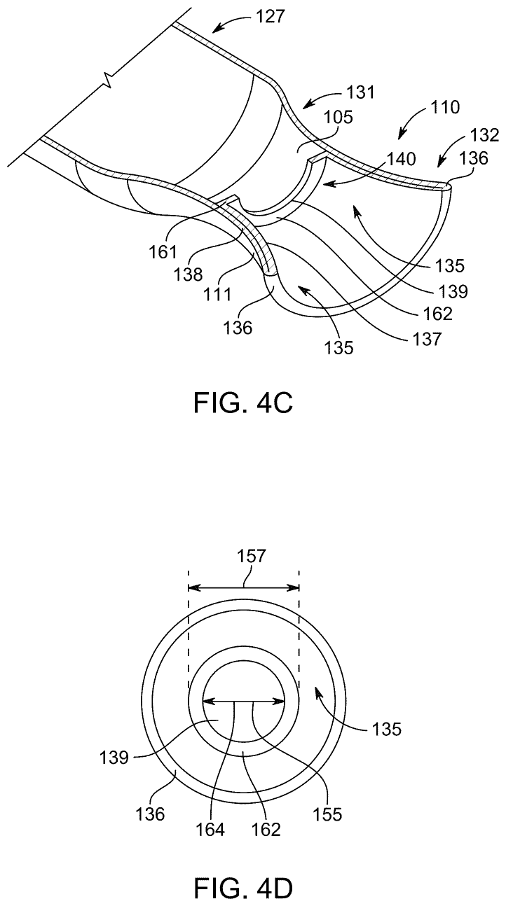

Once the outer member or the inner member is formed by winding one or more layers of material to form a cylindrical paper segment, the grip region 110 may be formed. The grip region 110 provides an improved area about which the user may hold a carboard tampon applicator and dispense the pledget from the cardboard paper applicator. As illustrated in FIGS. 4A and 4B, the outer member 102 includes a grip region 110 having a concave surface extending about the longitudinal axis 108. The portion of the grip region 110 having the concave surface is the gripping portion 111. The grip region 110 has a concave surface having a radius of curvature extending from an upper grip region 131 to a lower grip region 132, radius of curvature MD. The grip region 110 has a radius of curvature about the direction perpendicular to the longitudinal axis, the radius of curvature CD. The radius of curvature MD may be from about-5 mm to about-25 mm or from about-10 mm to about-20 mm. The radius of curvature CD may be from about 3 mm to about 8 mm or from about 4 mm to about 6 mm. The external outer member diameter 120 is less than at least a portion of the external grip region diameter 133. The external grip region diameter 133 changes in a direction parallel to the longitudinal axis 108 throughout the grip region 110. Further, the grip region has a variable cross-sectional area from the upper grip region to the lower grip region, as measured in a plane substantially perpendicular to the longitudinal axis 108.

The gripping portion 111 having the concave surface has a Gaussian curvature that is not equal to zero. The Gaussian curvature (K) is determined according to the following formula:

$$K = {k}_{1} * {k}_{2}$$

$$where {k}_{1} = 1 / {r}_{1} and {k}_{2} = 1 / {r}_{2} ,$$

and
r₁ is the radius of curvature of the grip region when measured in a plane that is coincident to the longitudinal axis 108 and r₂ is the radius of curvature of the smallest cross section of the grip region when measured in a plane that is orthogonal to the longitudinal axis 108.

The gripping portion has a gaussian curvature of from about-0.2 mm⁻² to about 0.4 mm⁻² or from about −0.1 mm⁻² to about 0.4 mm⁻² or from about-0.4 mm⁻² to about 0.4 mm⁻². The following table, Table 1, includes calculations of the gaussian curvature for various sized tampon applicators. The size of the tampon applicator is determined by the external diameter of the intermediate region of the outer member.

TABLE 1

| Diameter Intermediate | Radius of | Radius of | Gaussian |
|---|---|---|---|
| Region | Curvature | Curvature | Curvature |
| (mm) | MD (mm) | CD (mm) | (mm⁻²) |
| 12 | −20.6 | 4.1 | −0.012 |
| 14 | −17.9 | 4.8 | −0.012 |
| 16 | −13.8 | 5.2 | −0.014 |
| 18 | −11.1 | 5.3 | −0.017 |

Referring to FIG. 4C, which is a cross-section view of the grip region of the outer member, the grip end region includes an inverted region 135. The inverted region 135 is formed by folding a portion of the wound cylindrical segment such that a portion of grip end region is folded forming a fold 136 such that a portion of the wall of the cylindrical segment is adjacent to the inner surface of the cylindrical paper segment. Stated another way, the wound one or more layers forms a cylindrical segment or a cylindrical paper segment, and an end of the cylindrical paper segment that will form the grip region is inverted forming a fold 136. Thus, a portion of the grip region includes a first, internal ply 137 and a second, external ply 138 position adjacent one another. It is to be appreciated that a ply may include one or more layers of material. An internal portion of the inverted region 135 opposite the fold 136 is used to form a pusher opening 139. To form the pusher opening, an inverted edge region 140 of the internal portion of the inverted region 135 is folded, at an opening fold 161, to form an opening rim 162 and the pusher opening 139. The opening rim 162 extends from the fold 161 to the pusher opening 139. The pusher opening may have a pusher opening cross-sectional width 155 measured perpendicular to the longitudinal axis 108 that is less than the internal grip region cross-sectional width 157 measured perpendicular to the longitudinal axis 108, as illustrated in FIG. 4D. It is to be appreciated that the inverted edge region 140 may not be folded and the pusher opening 139 will be the same shape and width as the internal surface of the grip region. However, by folding the inverted edge region 140, the pusher opening 139 may be customized, including the shape and size of the opening, while being internal the cylindrical paper segment of the outer member. The pusher opening 139 may be circular, as illustrated in FIG. 4D, or non-circular, such as elliptical, square, rectangular, triangular, and other non-circular shapes. Thus, the pusher opening 139 may have a largest dimension 164, which is the longest dimension measured at two points in a plane perpendicular to the longitudinal axis 108. The largest dimension 164 may or may not be the same as the pusher opening width and/or pusher diameter depending on the shape of the pusher opening. The largest dimension 164 of the pusher opening 139 may be from about 3 mm to about 20 mm or from about 7 mm to about 10 mm or from about 6 mm to about 8 mm.

The pusher opening 139 may be positioned internally less than about 8 mm or less than about 5 mm or less than about 3 mm or less than about 2 mm or less than about 1 mm from the smallest cross-sectional area of the grip region measured parallel to the longitudinal axis 108. The smallest cross-sectional area of the grip region is measured in a plane substantially perpendicular to the longitudinal axis of the cylindrical paper segment. The pusher opening 139 may be positioned internally less than about 8 mm or less than about 5 mm or less than about 3 mm or less than about 2 mm or less than about 1 mm from the plane where the outer circumferential surface of the grip region is smallest as measured parallel to the longitudinal axis 108.

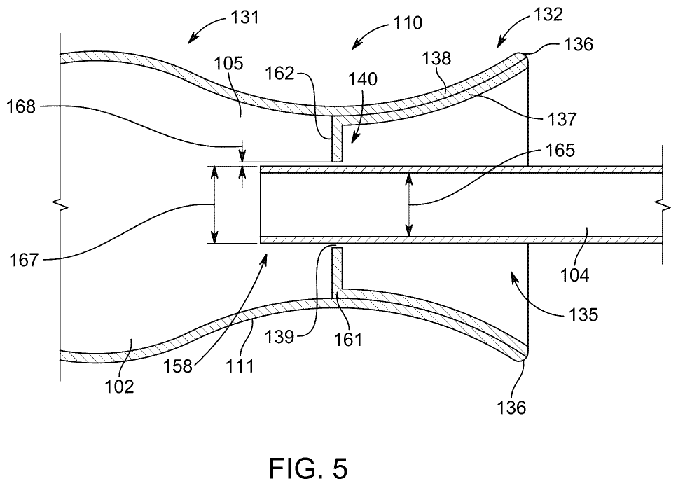

With reference to FIG. 5, the inner member 104, also referred to herein as a pusher, may extend through the pusher opening 139. More specifically, the first end region 158 of the pusher 104 extends through the pusher opening 139 in a pre-dispensing configuration (while the pledget is disposed within the outer member and prior to expelling the pledget from the outer member). The inner member 104 includes an internal pusher largest dimension 165, which may be the internal diameter, and an external pusher largest dimension 167, as measured perpendicular to the longitudinal axis 108. The internal pusher largest dimension 165 may be zero, where the pusher is manufactured from a solid piece of material and is not hollow, to about 19 mm. The external pusher dimension may be from about 1 mm to about 20 mm. The external pusher dimension may be different based on where the external pusher dimension is measured. For example, with reference to FIG. 5, the pusher illustrated in FIG. 5 has a first end region 158 and an intermediate region 159 that is similarly sized. However, as illustrated in FIGS. 2C and 2D, the first end region 158 and/or the second end region 160 may have a different dimension than the intermediate region 159. The dimensions, measured perpendicular to the longitudinal axis 108, of the first end region 158 and the second end region 160 may be greater than the intermediate region 159 to aid in engaging the pledget, provide an relatively larger surface area for the consumer to engage on the pusher, and to prevent the pusher from unintentionally disengaging the outer member through either the insertion region and/or the grip region.

The pusher, also referred to herein as an inner member, is sized as to operatively engage with the pusher opening. The pusher may be sized so that at least the intermediate region of the inner member may slidably engage the pusher opening and, thus, traverse in a first direction and a second, opposite direction, through the pusher opening. Creating relatively high interaction between the inner member and the pusher opening may create relatively higher friction between the two surfaces. Thus, the interaction of the pusher opening and the pusher should be controlled to allow for case of movement of the pusher to allow the user to comfortably operate the tampon applicator. Coatings, such as those disclosed herein, may be used to allow for case of movement of the pusher through the pusher opening. A coating may be disposed on the pusher opening and/or the external surface of the pusher, also referred to herein as the inner member. Additionally, the gap 168 between the external surface of the pusher and the pusher opening should not be so large that the pusher is free to wobble within the opening allowing for misalignment when engaging the pledget and leading to difficulty in operating the tampon applicator. Also, the gap 168 may not be so large as to allow the pusher to easily disengage or separate from the outer member. To provide a consumer preferable configuration of the pusher and the pusher opening, an outer perimeter of the pusher/inner member may be from about 80% to about 100% of a perimeter of the pusher opening. To provide a consumer preferable configuration of the pusher and the pusher opening, a cross sectional area as bounded by the perimeter of the pusher/inner member may be from about 80% to about 100% of a cross sectional area as bounded by the perimeter of the pusher opening. The gap 168 or distance between the pusher opening and the external surface of the pusher should be less than about 5 mm or less than about 3 mm or less than about 1 mm.

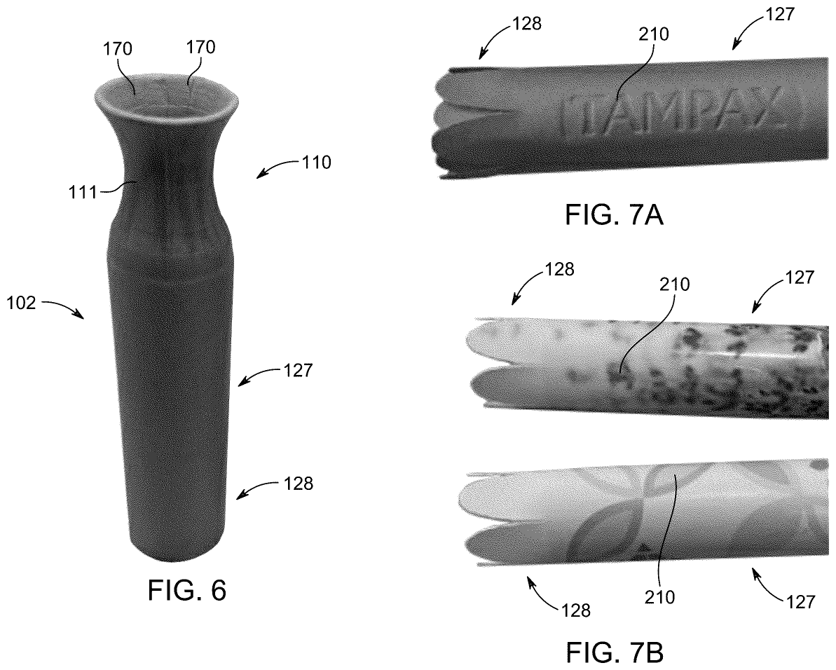

As previously discussed, each of the outer member and the inner member may be made from a cylindrical paper segment, which includes one or more layers made from cellulosic material. Duc to the nature of the cellulosic material, to form a grip region, the cylindrical segment needs to be compressed or formed. When compressing cellulosic material, such as paper, the material moves creating both macro-fold and micro-folds, such as illustrated in FIG. 6. The micro-folds are folds that occur that are not visually discernable and the macro-folds are folds that occur that are visually discernable. A visually discernible difference exists if an observer in ordinary indoor lighting conditions (20/20 vision, lighting sufficient to read by, for example) can visually discern a fold. However, at least a portion of the micro-folds may be detectable when light is used that is transmitted through the inner member and/or the outer member. FIG. 6 is a photograph of an outer member, which does not include petals at the insertion end, made from one or more layers of paper, and including a grip region as described herein, and the grip region includes one or more macro-folds 170 and one or more micro-folds, not visible in the photograph. It is to be appreciated that the one or more macro-folds may be present on only the inverted region of the grip region or on both the gripping portion and the inverted region.

The outer member and/or the inner member may include an indicium. The indicium 210 may be printed indicium and/or tactile indicium, which includes one or more textures. The tactile indicium is perceivable by touch of the human hand, human fingers respectively, and conveys information to the consumer regarding the consumer product. The tactile indicium may be a raised or indented portion of the inner or outer member. For example, the tactile indicium may comprise a series of raised elements, which together create a raised symbol that is perceivable by touch of the human hand and which conveys information with respect to the consumer product. Alternatively, the tactile indicium may comprise a series of indented elements, which together create an indented symbol that is perceivable by touch of the human hand and which conveys information with respect to the consumer product. The tactile indicium may be raised above the base level of the outer surface and/or indented below the base level of the outer surface. The tactile indicium may include elements of any suitable shape. The tactile indicium may include one or more element of square shape, rectangular shape, triangular shape, polygonal shape, (super) elliptical shape, circular shape, or a mixture thereof.

Such tactile indicium may be formed through the same process used to form the grip region using the compression device or in a subsequent or proceeding separate process. In still other example embodiments, additional material may be disposed on the external surface of the outer member and/or the inner member. Such additional material may include printed material (e.g., graphics, alphanumeric characters, etc.) or a material additive to create a texture.

The tactile indicium may include or may be braille text according to the 2010 ADA Standards for Accessible Design section 703.3. In particular, the tactile indicium may be a combination of braille text according to the 2010 ADA Standards for Accessible Design section 703.3 and a symbol specific to the type of absorbent article. As such, the tactile indicium is accessible to consumers not able or willing to read braille text as discussed before, while conveying extended information for consumers via the braille text.

The tactile indicium may include a brand identifier and/or artwork. A brand identifier may exemplarily be a tradename or brand-name, either in Braille text or as elevated text, a brand logo, an icon and/or character associated with a brand or company by the consumer. For example, as shown in the photograph of FIG. 7A, a brand name, such as TAMPAX, may be an indented element that is embossed into the outer member of the tampon applicator.

The package may include a printed indicium conveying the same or different information as the tactile indicium. The printed indicium may be a word, symbol, color, bar code, QR code or mixtures thereof. The printed indicium may be the same symbol as the tactile logo. The printed indicium may not be raised above the base level of the outer surface or indented below the base level of the outer surface. Alternatively, the printed indicum may at least partially overlap with the tactile indicium.

In one example, the printed indicum may at least partially, preferably completely, overlap with the tactile indicium forming an overlapping area. The tactile logo may be raised above the base level of the outer surface, and the printed indicium may exhibit a color at the maximum height of the overlapping area. The color at maximum height of the overlapping area may be different than the color of the base level of the outer surface. Alternatively, the tactile logo may be indented below the base level of the outer surface, and the printed indicium may exhibit a color at the maximum depth of the overlapping area. The color at maximum depth of the overlapping area may be different than the color of the base level of the outer surface. The printed indicum may be continuous about the cylindrical segment. In an example, as shown in the photograph of FIG. 7B, printed indicium may be disposed in a predetermined pattern about the outer surface or continuously about the outer surface. The printed indicium may include one or more colors.

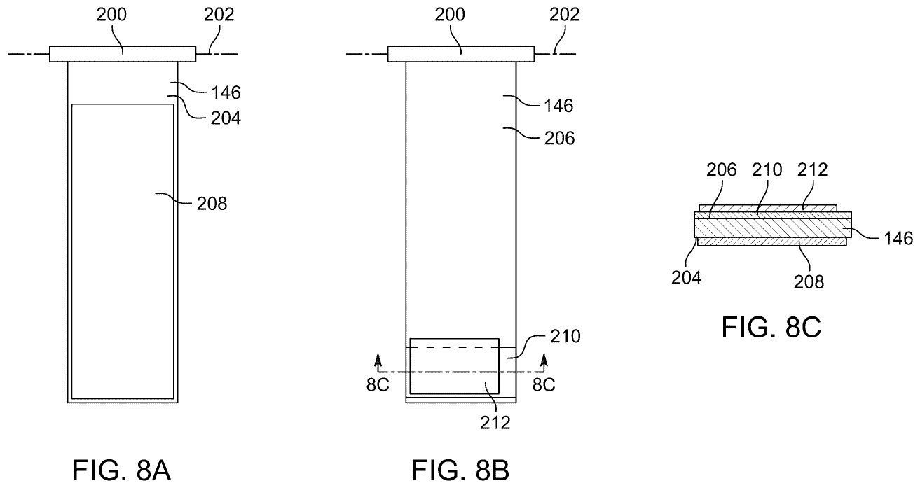

The method of forming the outer member includes winding one or more layers of material about a winding axis, as previously discussed. Prior to winding, one or more layers of material that will be used to form the cylindrical segment or cylindrical paper segment may be provided either as a roll of material or as individual sheets of material. Optionally, one or more coatings and/or indicium may be applied to the one or more layers prior to winding. A coating may include an adhesive, a glide coating, such as wax or hot melt, or a coating that acts as both an adhesive to bind the one or more revolutions of material and/or the seam, such as previously discussed herein, and a glide coating to reduce friction and case insertion and removal of the applicator. With reference to FIGS. 8A to 8C, the one or more layers of material 146, which may be paper, may be advanced to a winding apparatus 200. The winding apparatus 200 rotates about a winding axis 202 to wind the one or more layers either convolutedly or spirally. Prior to winding, at least one of a coating, an adhesive, and indicium may be disposed on one or more sides of the one or more layers of material 146. For example, the one or more layers of material 146 may have a first side 204 and a second side 206. An adhesive 208 may be disposed on the first side 204 of the one or more layers to bind the revolutions of the one or more layers as the material is wound. The adhesive may be applied to the entire surface of the side of the material layer or to a portion of the side of the material layer. The adhesive may be applied in a pattern, continuously, or intermittently. The adhesive may be a material as previously discussed herein. FIG. 8A illustrates a first side 204 of a material layer having adhesive 208 disposed thereon and extending continuously across the surface of the first side. However, the adhesive may not be applied on the surface of the first side where the one or more layers directly contacts the winding apparatus so that the material layer does not adhere to the winding apparatus or transfer adhesive to the winding apparatus. Additionally, adhesive may be applied so that it is not present in the insertion region.

Similarly, at least one of a coating and an indicium may be applied to the second side 206 and/or the first side 204 of the one or more material layers. The coating and/or the indicium may be applied in a pattern, continuously, intermittently, or over a portion of the one or more material layers. As illustrated in FIG. 8B, indicium 210, which may be visual and/or tactile, such as a printed graphic and/or alphanumeric print, and/or tactile, such as an emboss and/or deboss, may be disposed on the second side 206 of the one or more layers. Alternatively or additionally, a glide coating may be disposed on the second side 206 of the one or more layers. The glid coating may be applied such that the glid coating is only present on the outer or exterior surface of the outer member. The glide coating may be disposed on what will become the outer or exterior surface of the outer member after being wound. The glide coating may be applied in the same area as the indicium or may overlap a portion of the indicium. The glide coating may be applied over the entire outer or exterior surface of the outer member or a portion of the outer or exterior surface of the outer member. For example, the glide coating may be disposed on the outer or exterior surface except for the grip region so that the grip region has a different tactile feel than the remainder of the outer member and may be easier to grip given the increased friction over the portion of the outer member with the glide coating. This may also be a visual signal to the user that the grip region will be easier to hold on to given its less glossy appearance when the glide coating is not applied or applied in a different manner to the grip region. Similarly, the glide coating may signal to the user that the outer member has a glossy and smooth exterior surface which allows for case of insertion and removal during use.

A glide coating may also be applied to an interior surface of the outer member so that the pledget slides within the outer member and can be expelled with an expulsion force of less than about 1200 gf or less than about 800 gf or less than about 650 gf. However, it is to be appreciated that the material of the one or more layers may have a surface that is relatively smooth and does not require any coating to expel the pledget with the desired expulsion force as discussed herein. FIG. 8C illustrates a cross-sectional end view of the one or more layers including an adhesive 208, a glide coating 212, and indicium 210. It is to be appreciated that not all of these coatings may be disposed on the one or more layers, and also, additional coatings may be applied to the one or more layers. In one example, a paper layer may be advanced toward the winding apparatus and a PVA-adhesive may be applied to a surface of the paper layer and a polyethylene coating may be applied to a surface of the paper layer prior to winding. In another example, a paper layer may be advanced toward the winding apparatus, and a starch adhesive may be applied to a surface of the paper layer and a wax coating may be applied to a surface of the layer prior to winding.

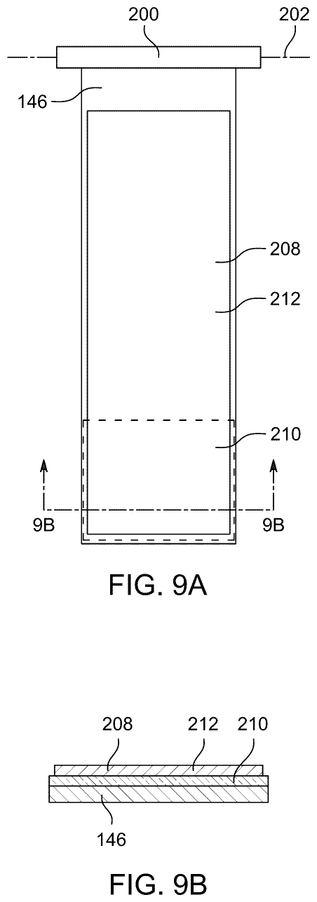

Referring to FIGS. 9A-9B illustrates another example of one or more layers including one or more coatings. As illustrated, a coating that acts as both an adhesive 208 and a glide coating 212 may be applies to the first side or the second side of the one or more layers. A coating that acts as both an adhesive and a glide coating may include, for example, polyethylene or polybutylene succinate, or similar materials. Further, a coating that acts as both an adhesive and a glide coating may be applied in a first state and then gets activated to transform into a second state. For example, a coating may be applied that immediately acts to form a glossy, smooth outer surface but is not an adhesive. However, upon subjecting the coating to heat, radiation, UV, moisture, fluid, and/or some other energy source, the coating becomes an adhesive and joins to that which it interacts with.

It is to be appreciated that any number of coatings may be applied to the one or more layers prior to winding and the placement of the coatings may be such that the coating is positioned between the revolutions of the one or more layers as they are wound and/or on the exterior/interior surface of the outer member. An exterior or interior coating may also be applied after winding the one or more layers.

It is also to be appreciated that a coating and an indicium are optional and need not be applied prior to or after winding the cylindrical segment. As previously discussed, moisture may be applied to form the cylindrical paper segment. The moisture may be directly applied to at least a portion of the one or more layers or the environment in which the cylindrical paper segment is formed may be controlled such that the moisture content of the one or more layers is increased, such as by humidification of the manufacturing environment.

The one or more layers may be wound from a continuous roll of material or the one or more layers may be cut to a specific length prior to or while winding. If the one or more layers are wound continuously, such as when spirally winding, the continuous cylindrical tube will then be cut into cylindrical paper segments or cylindrical segments to form the outer member. For convolute winding, the one or more layers are cut into discrete segments and then wound or the winding may begin from a larger roll but after the desired number of revolutions, the one or more layers may be cut so that a scam may be formed on the cylindrical tube. It is to be appreciated that when convolutedly winding, more than one outer member or cylindrical paper segment may be formed in a longer cylindrical tube and then the tube may be cut to form the individual cylindrical paper segments.

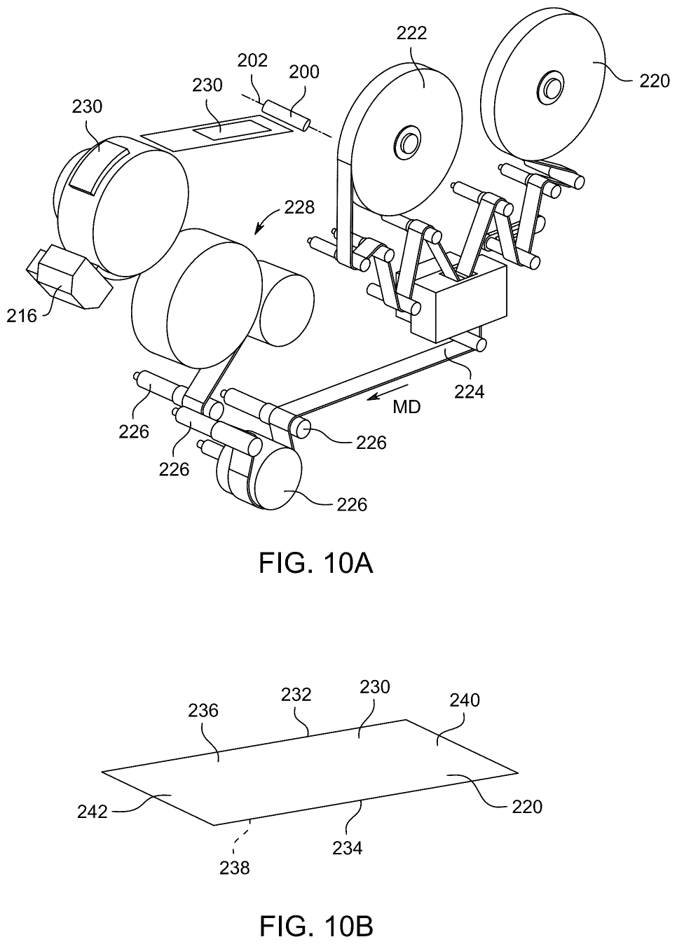

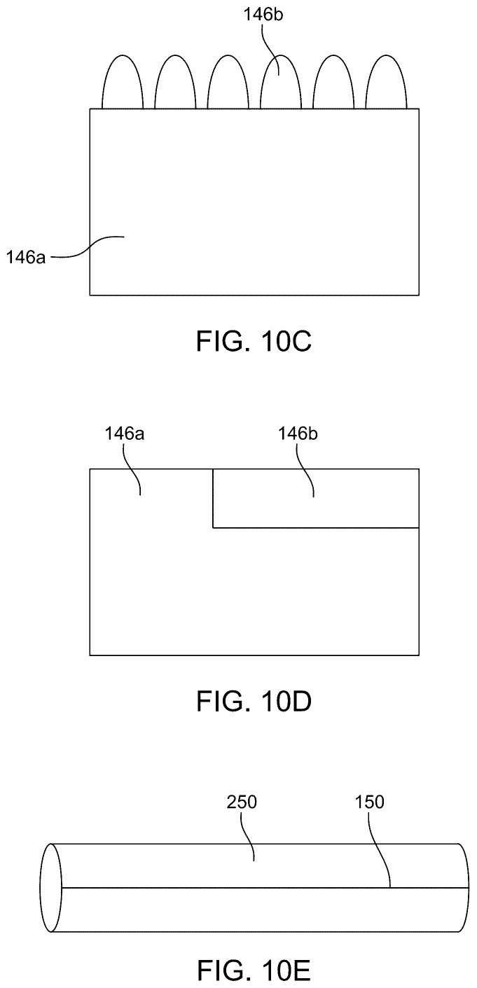

FIG. 10A illustrates an example of an apparatus and process that may be used to form the cylindrical segment which may form the outer member or inner member. However, for this discussion, the focus will be on the outer member but the following is also applicable to the inner member. In this embodiment, two rolls of material, a first roll of material 220 and a second roll of material 222, are provided. It is to be appreciated that one or more rolls of material may be provided. The individual rolls may include one or more layers of material. Material is unwound from the first roll of material 220 and material is unwound from a second roll of material 222. The material from the first roll of material may be combined with the material from the second roll of material to form a multi-layer material web 224. The multi-layer material web 224 may be two or more layers of material. However, a single layer of material may be unwound and advanced. The multi-layer material web 224 is advanced in a machine direction MD, which is the direction in which the web advances to downstream processes. The multi-layer web 224 may be advanced over one or more rollers 226 and to a cutting device 228. The one or more rollers 226 may be used to appropriately position the web 224 within the cutting device 228 and to advance the discrete segment or discrete paper segment 230 from the cutting device 228.

The discrete paper segment or discrete segment 230 continues to advance in a machine direction MD. The discrete segment 230 includes a first side edge 232, a second side edge 234, a first surface 236, a second surface 238, a leading edge region 240, a trailing edge region 242, as illustrated in FIG. 10B. The discrete segment 230 may traverse such that at least one of the first side edge 232 and the second side edge 234 or portions thereof are substantially parallel to the machine direction or the direction of advancement of the discrete paper segment. The discrete segment 230 may traverse such that at least one of the leading edge region 240 and the trailing edge region 242 or portions thereof are substantially perpendicular to the machine direction or the direction of advancement of the discrete paper segment. The second surface 238 of the discrete segment may be disposed on a transfer device, such as a belt, conveyor, and/or roll. It is to be appreciated that the discrete segment may have a non-rectangular shape or one or more of the individual layer may have a nonrectangular shape. For example, a first layer may have a rectangular shape and a second layer may have a cut-out or portion of the layer that is removed such that the second layer does not have a rectangular or square shape. The cut-out of portion of the layer that is removed may be about the perimeter of the discrete segment or an internal portion of the discrete segment. For example, as illustrated in FIG. 10C, a first layer 146a may be a substantially rectangular layer and a second layer 146b may include one or more cutouts that will form petals when the discrete segment is wound. In another example, as illustrated in FIG. 10D, a first layer 146a may include a cutout along the perimeter that allows for a portion the second layer 146b to be visible. Cutouts may allow for the number of revolutions of layers in a given region of the tampon applicator to be reduced, such as in the insertions region where petals may be formed after the discrete segment is wound.

Optionally, the discrete paper segment may be advanced to a coating device 216. The coating device 216 may be used to apply one or more coatings such as those previously discussed herein. The coatings may be applied prior to cutting the web of one or more layers of material into discrete segments. The coatings may be applied after the discrete segment is wound.

The discrete paper segment is advanced to a winding apparatus 200. The winding apparatus 200 rotates about a winding axis 202 and is configured to wind the discrete segment 230. The first surface 236, which may include one or more coatings as previously discussed, engages the winding apparatus 200, and the discrete segment is wound to form a cylindrical paper segment or cylindrical segment 250, such as illustrated in FIG. 10E. The cylindrical paper segment 250 may include an external scam 150 and an internal scam.

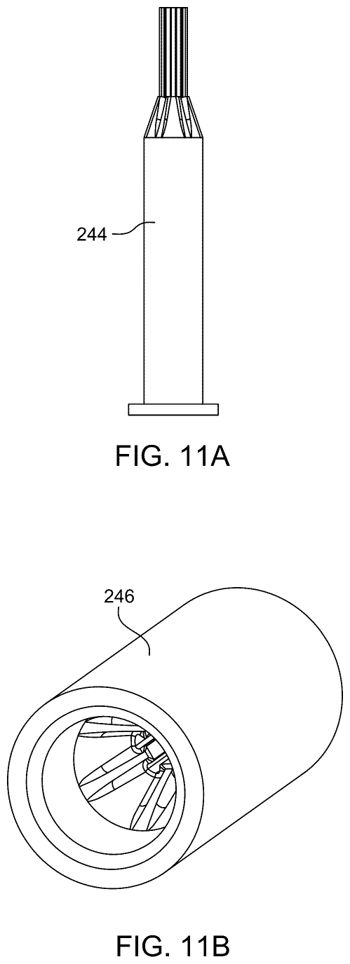

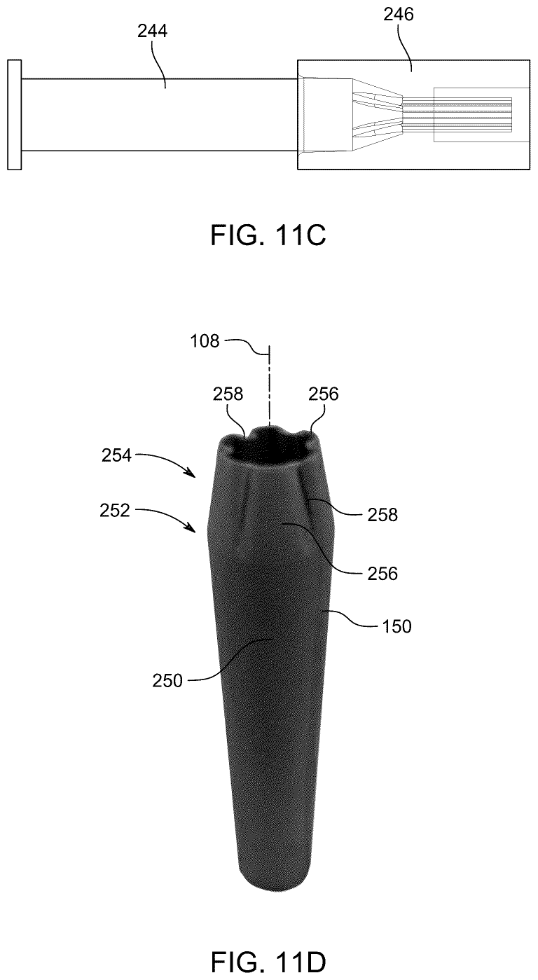

The formed cylindrical paper segment or cylindrical segment 250 may be advanced to additional downstream processes to form the grip region of the outer member. In some embodiments, the cylindrical segment 250 is advanced to a pleating device. The pleating device includes a pleating mandrel 244 and a pleating die 246, such as illustrated in FIGS. 11A-11C. A first end region 252 of the cylindrical segment 250 may be placed about the pleating mandrel 244 and then placed within the pleating die 246 to form one or more pleats 254. The pleats 254 may include a plurality of ridges 256 and valleys 258 extending about the longitudinal axis 108. The formation of the one or more pleats 254 is based on the design of the pleating mandrel 244 and pleating die 246. Each of the pleating mandrel 246 includes at least two raised portions and the pleating die 246 includes at least two recessed portions that are configured to operatively engage the at least two raised portions of the pleating mandrel. A portion of the first end region 252 is displaced and formed based on the at least two raised portions of the pleating mandrel and the at least two recessed portions of the pleating die. Pressure is applied to the pleating mandrel as the pleating mandrel engages the pleating die. It is also to be appreciated that heat and/or moisture may be applied to the cylindrical segment to aid in formation of the one or more pleats 254. The pleats 254 result in the first end region 252 tapering toward the longitudinal axis 108 or, stated another way, the pleats converge toward the longitudinal axis 108, such as illustrated in FIG. 11D. It is to be appreciated that forming the pleats 254 is optional to form the grip region. However, it has been found that by pleating the first end region 252, the one or more layers of the cylindrical segment may be formed more easily into the grip region as discussed herein. More specifically, by first pleating the grip region, the one or more layers are less prone to tearing during formation of the grip region. Also, less force may be needed to form the grip region because the one or more layers are already partially deformed and lines along which the layers move are provided by the ridges and valleys. It is to be appreciated that the pleats 254 may be formed with only a pleating die 246. To form the pleats 254 a pleating mandrel 244 is optional.

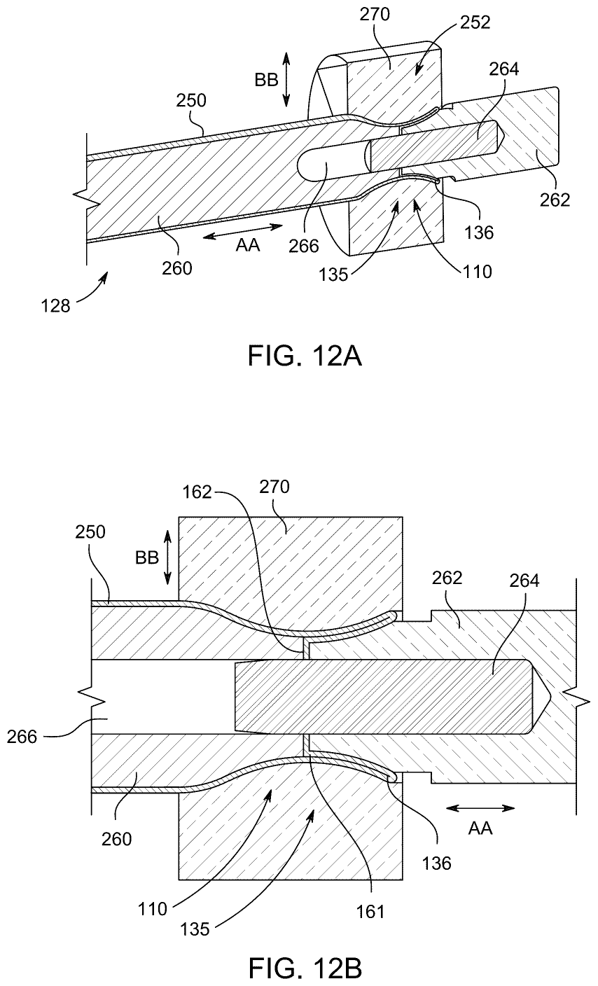

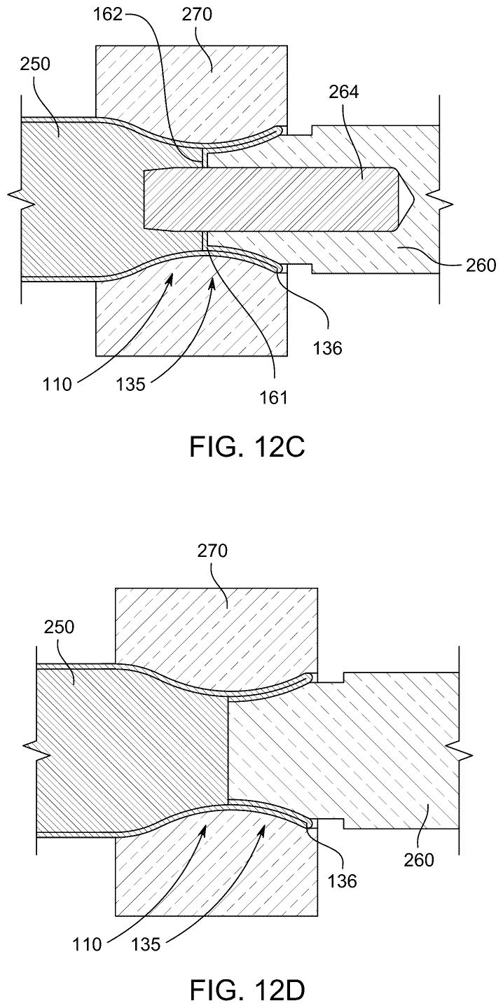

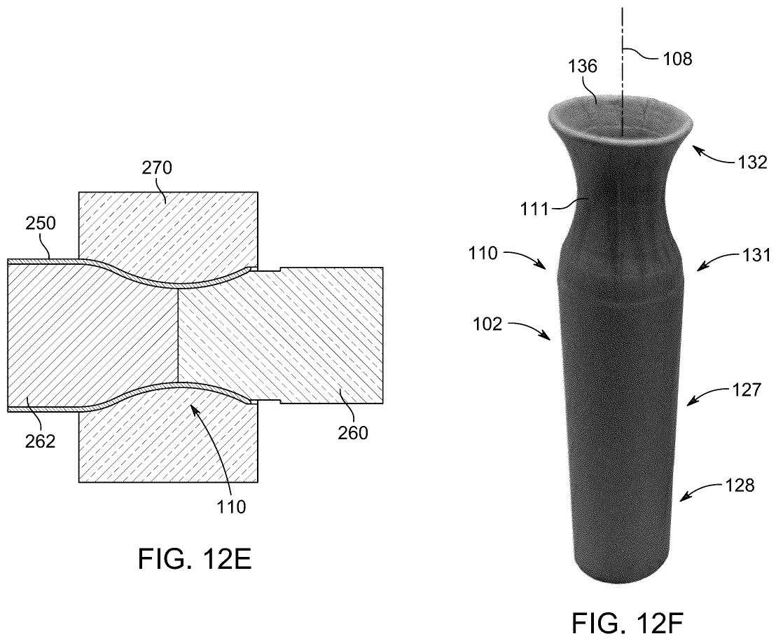

Referring to FIGS. 12A-12B, the grip region 110 may be formed by positioning a first mandrel 260 within the interior of the cylindrical segment 250. The first mandrel 260 may enter the cylindrical segment 250 through the insertion end region 128 and be positioned such that at least a portion of the first mandrel 260 is disposed in the grip region 110 or what is to be the grip region. A second mandrel 262 may be configured to engage the rearward end of the cylindrical segment. The second mandrel 262 is positioned such that only a portion of the second mandrel 262 is disposed in the grip region or what is to be the grip region. At least one of the first mandrel 260 and the second mandrel 262 support a forming rod 264. The forming rod 264 traverses with only one of the first mandrel 260 or the second mandrel 262. As illustrated in FIGS. 12A and 12B, the forming rod 264 is joined to the second mandrel 262 such that the forming rod 264 traverses with the second mandrel 262. The first mandrel 260 includes an opening 266 through which the forming rod may extend or slidably engage and be supported (such as to prevent deflection) during formation of the grip region, but the forming rod 264 does not traverse with the first mandrel 260. The opening in the first mandrel 260 may merely provide a place for the forming rod to extend. The forming rod 264 may have any desired cross-section shape. The cross-sectional shape of the forming rod may be circular or non-circular such as elliptical, square, rectangular, triangular, or polygonal shaped. It is also to be appreciated that the cross-sectional shape of the forming rod may be the same as the cross-sectional shape of the pusher so that the pusher is stabilized when positioned within the outer member. Alternatively, the cross-sectional shape of the pusher may be different than the cross-sectional shape of the forming rod. For example, the cross-sectional shape of the pusher may be circular and the cross-sectional shape of the forming rod and thus, the pusher opening may be non-circular. The cross-sectional shape of the forming rod will be the shape of the pusher opening in the tampon applicator.

To form the grip region 110, as illustrated in FIGS. 12A and 12B, the first mandrel and the second mandrel are configured to traverse in a first direction that is substantially parallel to the longitudinal axis of the cylindrical segment, indicated by arrows AA. The second mandrel 262 traverses toward the first mandrel 260. The second mandrel 262 engages the pleats 254 or first end region 252 of the cylindrical segment and invert the pleats 254 and/or the first end region 252 by pushing a portion of the first end region 252 inside the interior of the cylindrical paper segment. A portion of the first end region is positioned adjacent to the inner surface of the cylinder paper segment such that a wall of the first end region is disposed on an inner surface of the cylindrical paper segment. A fold 136 is formed in the first end region, such as previously discussed. The first mandrel and the second mandrel traverse until the once pleated region is inverted forming an inverted region 135. A fold 136 is formed in the first end region, such as previously discussed. The first and second mandrels continue to traverse towards one another forming the opening fold 161 and the opening rim 162. The opening rim 162 forms about the forming rod to form the pusher opening 139.

In some alternative embodiments, the pusher opening may be formed without the forming rod. The grip region may be formed as previously discussed but with or without the forming rod. Separately or in addition to the use of a forming rod, a pusher opening at or near an end region can be cut to create a pusher opening. This opening could be cut using a variety of techniques including but not limited to a matching shear cut die, crush cut die, or laser. This pusher opening whether formed by a forming rod or by cutting or by some combination thereof may be formed in a variety of shapes including but not limited to circular, elliptical, square, triangle, or cross. The pusher opening may be cut to any desired shape such that the pusher opening supports the inner member.

Still referring to FIGS. 12A and 12B, either while the first mandrel and the second mandrel traverse towards one another or after the first mandrel and the second mandrel are operatively engaged, a compression device 270 engages the exterior surface of the cylindrical segment in the grip region. The compression device 270 may include two or more chucks that operatively engage and substantially surround the exterior surface of the cylindrical segment in the grip region. The compression device 270 is configured to traverse in a direction substantially perpendicular to the longitudinal axis of the cylindrical segment, such as indicated by arrow BB. The compression device 270 may have a shaped surface that pairs with the surface of the first and/or second mandrel to from the radius of curvature of the grip region, as previously discussed herein. Each of the surface of the chucks of the compression device and/or the first and second mandrel may include raised portions that impart a tactile indicium to the grip region upon formation. Each of the chucks may apply the same pressure or variable pressure to the cylindrical segment to form the grip region. The chuck may apply a pressure of greater than about 25 psi or greater than about 150 psi or greater than about 250 psi or greater than about 500 psi or greater than about 1000 psi or from about 250 psi to about 5000 psi or from about 500 psi to about 2000 psi or from about 200 psi to about 1000 psi. It is to be appreciated that heat may be applied to the compression process, but it is not necessary to form the grip region. Heat may be applied using steam and/or heated chucks. Compression of the chucks against the first and second mandrel forms the grip region 110, such as illustrated in FIG. 12F. It is also appreciated that heat and/or steam and/or moisture may be added to the cylindrical segment prior to forming the grip region to aid in deforming the cylindrical segment to form the grip region.

The grip region may be formed with only a single, first mandrel 260 and a forming rod 264, such as illustrated in FIG. 12C. Similar to the above, the first mandrel and the forming rod traverse within the first end region of the cylindrical segment resulting in inversion of a portion of the first end region. The compression device then applies pressure to the external surface of the cylindrical segment to form the grip region, as previously discussed herein.

The grip region may be formed with a single, first mandrel and no forming rod, such as illustrated in FIG. 12D. The first mandrel would invert a portion of the first end region as previously discussed and the pusher opening would then become the narrowest cross sectional opening within the interior of the grip region. The inversion of the first end region would from a fold 136 as previously discussed. Also, the compression device would apply pressure to the exterior surface of the cylindrical segment to form the grip region, as previously discussed herein.

It is also to be appreciated that the grip region may not include an inverted portion. As illustrated in FIG. 12E, a first mandrel 206 and optionally a second mandrel 262 may be used to from the grip region. Each of the first mandrel and optionally the second mandrel 262 may be inserted into an interior of the cylindrical segment and the compression device may compress the outer surface forming the grip region, as previously discussed herein. The radius of curvature of the grip region is formed by the compression device and the mandrels and is shaped as previously discussed.

FIG. 12F illustrates an outer member including a grip region that has been formed by a cylindrical paper segment from one or more of the processes discussed herein. The grip portion 110 has a concave portion having a radius of curvature and a gaussian curvature as previously discussed. As illustrated in FIG. 12F, the grip region is relatively smooth and appears more plastic like. The folds in the applicator due to processing the cylindrical paper segment are relatively unnoticeable.

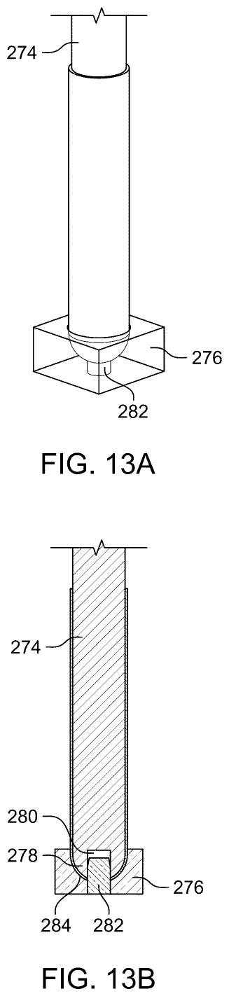

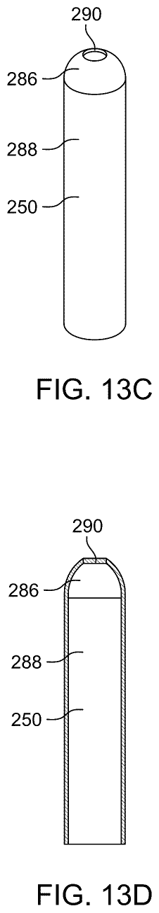

In an alternative embodiment, after pleating the first end region of the cylindrical segment, as illustrated in FIG. 11D, the pleated cylindrical segment may undergo an additional transformation prior to being inverted. In this alternative embodiment, the pleated cylindrical segment is advanced to a dome forming mandrel 274 and a dome forming die 276, such as illustrated in FIGS. 13A and 13B. The dome forming mandrel 274 includes a domed region 278 that defines an interior cavity region 280. The interior cavity region 280 is configured to receive a forming rod 282. The forming rod 282 is configured to slidably engage the interior cavity region. Alternatively, the interior region 280 may support a forming rod such that the forming rod is fixed within the interior cavity region and extends from the dome region toward the forming die. The dome forming mandrel 274 is configured to engage the dome forming die 276. The dome forming die 274 includes a domed forming surface 284 which is the same shape as the domed region and is configured to engage with the domed region 278. The dome forming die 276 may support a forming rod such that the forming rod is fixed within a region of the dome forming die and extends from the die toward the mandrel. Alternatively, the forming rod 282 is configured to slidably engage an interior region of the dome forming die. As illustrated in FIGS. 13A and 13B, the pleated cylindrical segment is slidably engaged by the dome forming mandrel. The dome forming mandrel is placed within the interior of the pleated cylindrical segment. The pleated cylindrical segment is disposed over the domed region. The dome forming mandrel including the cylindrical segment then engages the dome forming die to form a dome region 286 in the cylindrical segment or a domed cylindrical segment 288 such as illustrated in FIGS. 13C and 13D. The domed cylindrical segment 288 includes an opening 290, formed by the forming rod, and the portion of the domed region 286 surrounding the opening 290 has a convex surface having a Gaussian curvature (K) of from about 0.050 mm⁻² to about 0.010 mm⁻², which is calculated according to the formula previously disclosed herein.

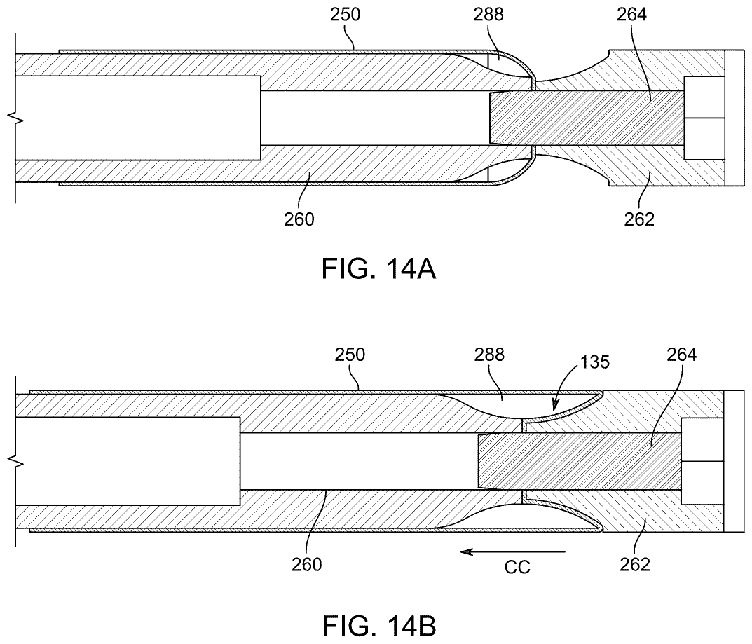

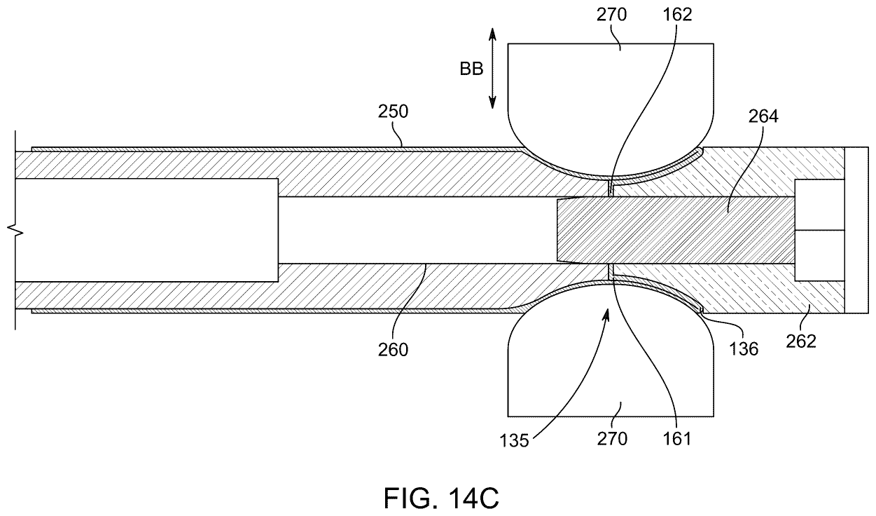

The domed cylindrical segment 288 may then be inverted, such as previously discussed. The domed cylindrical segment 288 may be advanced to a first mandrel 260 and a second mandrel 262, such as illustrated in Figured 14A-14C. The first mandrel 260 may slidably engage the domed cylindrical segment 288. The first mandrel 260 slides within the interior of the cylindrical segment 288. The second mandrel 262 engages the exterior of the cylindrical segment about the opening 290. A forming rod 264 is inserted through the opening 290 of the cylindrical segment 288. The forming rod 264 may be held by either the first mandrel or the second mandrel. The opposing mandrel, or the mandrel that does not hold the forming rod 264 includes a cavity configured to allow the forming rod to advance into during inversion of the cylindrical segment. As illustrated in FIG. 14A, the first mandrel 260 and the second mandrel 262 operatively engage the cylindrical segment about the opening and a forming rod extends through the opening. With the cylindrical segment remaining stationary or fixed, the first mandrel and the second mandrel advance in a direction away from the domed region or toward the end of the cylindrical segment opposite to the domed region, which is indicated by arrow CC in FIG. 14B. The first mandrel and the second mandrel move until the once domed region is inverted forming an inverted region 135. A fold 136 is formed in the first end region, such as previously discussed. The first and second mandrels traverse together forming the opening fold 161 and the opening rim 162. The opening rim 162 forms about the forming rod to form the pusher opening 139. It is also to be appreciated that the first mandrel and the second mandrel may remain fixed or stationary and the cylindrical segment is moved with respect to the first mandrel and the second mandrel.

In some alternative embodiments, the domed cylindrical segment 288 may be formed without the forming rod. The domed cylindrical segment 288 may be formed with a dome forming die 174 and a dome forming mandrel 274 that do not include a forming rod. The domed shape is formed and a hole at the end of the dome may or may not be present. Additionally or alternatively, the domed cylindrical segment may be inverted without the use of a forming rod. The inversion may be done by the first mandrel and the second mandrel.

With reference to FIG. 14C, a compression device 270 engages the exterior surface of the cylindrical segment to form the grip region. As previously discussed, the compression device 270 may include two or more chucks that operatively engage and substantially surround the exterior surface of the cylindrical segment in the grip region. The compression device 270 is configured to traverse in a direction substantially perpendicular to the longitudinal axis of the cylindrical segment, such as indicated by arrow BB. The compression device 270 may have a shaped surface that pairs with the surface of the first and/or second mandrel to from the radius of curvature of the grip region, as previously discussed herein. Each of the surface of the chucks of the compression device and/or the first and second mandrel may include raised portions that impart a tactile indicium to the grip region upon formation. Each of the chucks may apply the same pressure or variable pressure to the cylindrical segment to form the grip region. The chuck may apply a pressure of greater than about 25 psi or greater than about 150 psi or greater than about 250 psi or greater than about 500 psi or greater than about 1000 psi or from about 250 psi to about 5000 psi or from about 500 psi to about 2000 psi or from about 200 psi to about 1000 psi. Compression of the chucks against the first and second mandrel forms the grip region 110, such as illustrated in FIG. 14C. It is also appreciated that heat and/or steam and/or moisture may be added to the cylindrical segment prior to forming the grip region to aid in deforming the cylindrical segment to form the grip region.

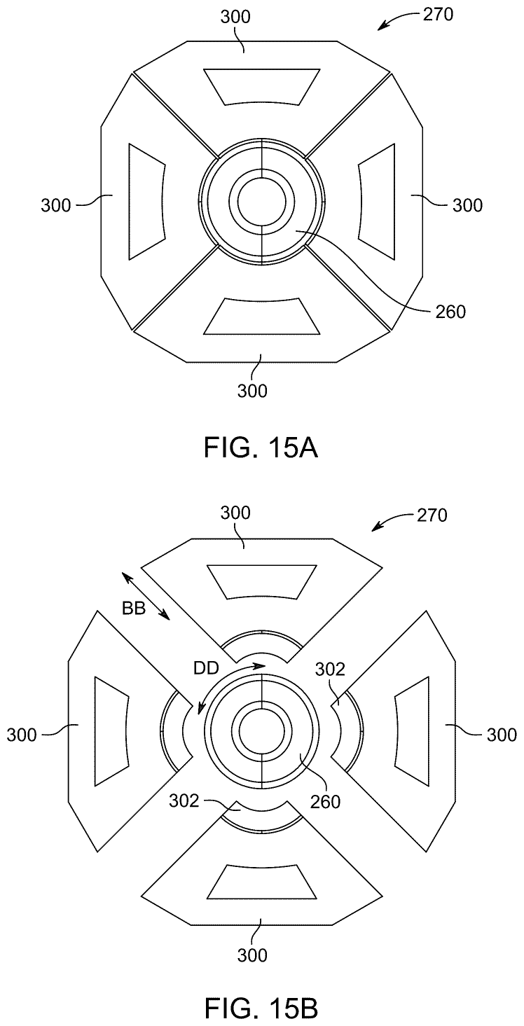

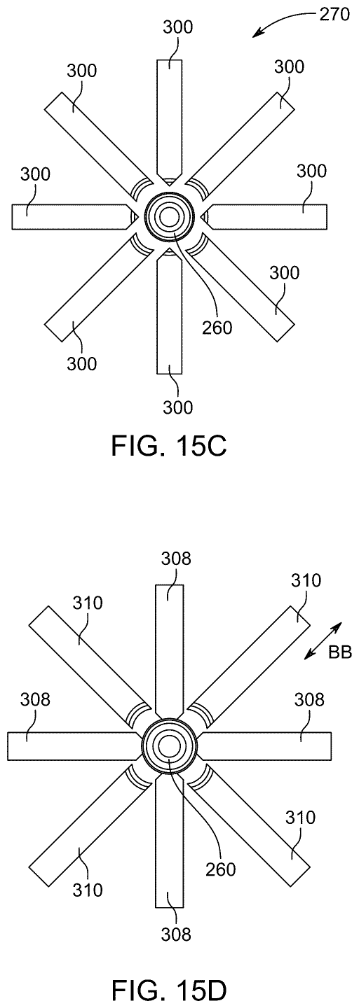

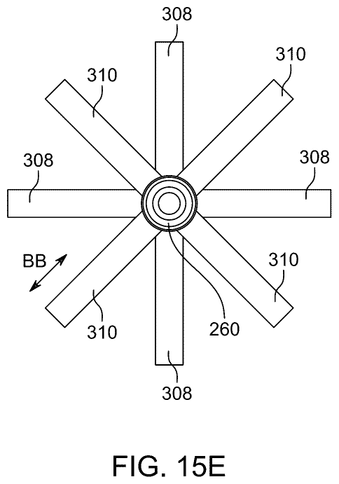

The configuration of the compression device 270 may allow for different surfaces features to be imparted to the grip region 110. The compression device 270 may include two of more chucks to form these surface features. The chucks generally pair with the shape of the mandrel to from the concave surface of the grip region. FIGS. 15A and 15B illustrate an example embodiment of the compression device 270 including four chucks 300. As previously discussed, the cylindrical segment is disposed about the mandrel 260 such that the mandrel extends through at least a portion of the interior of the cylindrical segment. The chucks 300 traverse toward and away from the mandrel 290 in a radial direction as indicated by arrow BB. Each of the chucks may apply the same pressure or different pressure when engaging the cylindrical segment to form the grip region. As illustrated in FIGS. 15A and 15B, the chucks 300 may each have a chuck surface 302. The chuck surface 302 may be a concave and/or convex surface. The chuck surface 302 may include raised portions to from one or more tactile indicium in the grip region of the cylindrical segment. FIG. 15A illustrated the compression device 270 in a closed configuration and FIG. 15B illustrates the compression device 270 in an open configuration. As illustrated in FIG. 15A, when the compression device 270 is in a closed configuration, the two or more chucks are configured to about one another and surround the cylindrical segment to form the grip region such as illustrated in FIG. 12F. It is also to be appreciated that the compression device 270 may be configured to compress the cylindrical segment multiple times. For example, the compression device 270 may be placed in the open configuration to allow the mandrel including the cylindrical segment to be placed within the compression device. The compression device may traverse radially inward and compress the cylindrical segment in a first compression sequence. Without laterally traversing the mandrel, the compression device may at least partially open and then radially traverse inward to compress the cylindrical segment in a second compression sequence. It is to be appreciated that the mandrel including the cylindrical segment may be moved between the first compression sequence and the second compression sequence. The mandrel including the cylindrical segment may be traversed in a lateral direction and/or a radial direction, such that the mandrel including the cylindrical segment is rotated about a central mandrel axis in either a first radial direction or a second radial direction, as indicated by arrow DD. Allowing the mandrel to radially rotate between the first and second compression sequence may provide a smoother grip region.

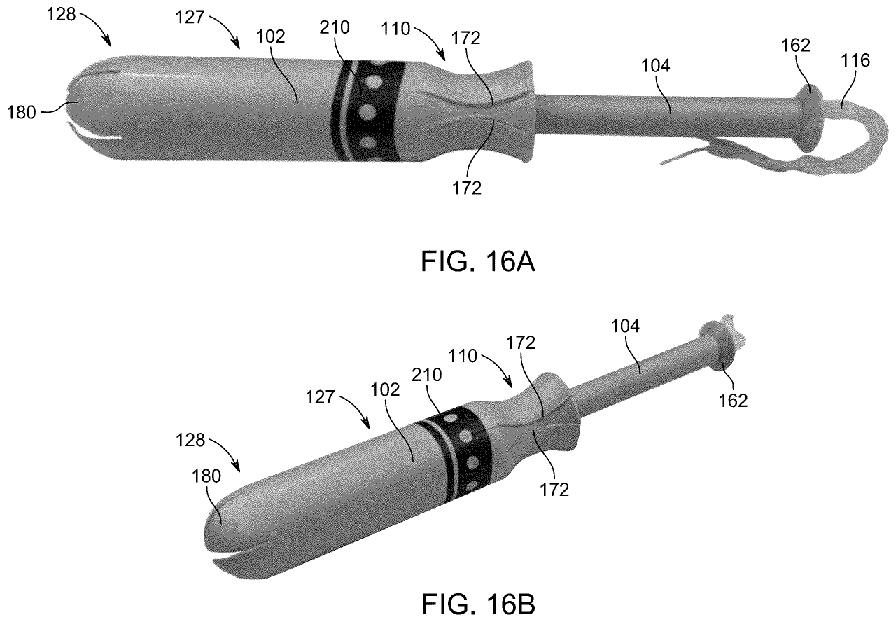

FIGS. 15C-15E illustrate another example configuration of a compression device 270. The compression device 270 may include each of the features and functions as discussed with respect to FIGS. 15A and 15B. Additionally or alternatively, FIG. 15C illustrates a compression device 270 including eight chucks 300 positioned in an open configuration about the mandrel. Of the eight chucks 300, four of the chuck 300 have first surface profile, which will be referred to as the first group of chucks 308, and the remaining four chucks 300 have a second surface profile, which will be referred to as the second group of chucks 310. The first surface profile is different from the second surface profile. As previously discussed, the chucks 300 traverse in a radial direction toward and away from the mandrel. As illustrated in FIG. 15D, the first group of chucks 308 may be configured to traverse and operatively engage the mandrel while the second group of chucks 310 remain stationary. Once engaged by the first group of chucks 308, the second group of chucks 310 may subsequently operatively engage the mandrel, such as illustrated in FIG. 15E. The chucks may be removed or traversed away from the mandrel in a similar sequential configuration, such as the first group of chucks being traversed away from the mandrel and then, subsequently, the second group of chucks, or the chucks may be traversed away from the mandrel such that all the chucks traverse at the same time. The pressure applied to the mandrel by the first group of chucks and the second group of chucks may be the same or different. By having one or more chucks have varying surface profiles and by traversing the chucks in some sequential pattern, such as by group, the surface of the grip region may be altered. For example, using the various profiles and sequence of movement as described with respect to FIGS. 15C-15E, the grip region 110 may include two or more ridges 172 as illustrated in FIGS. 16A and 16B. As previously discussed, the tampon applicator, as illustrated in FIGS. 16A and 16B, appears more plastic-like but is manufactured using paper. The tampon applicator 100 includes an outer member 102 including an insertion region 128 including petals 180, an intermediate region 127, and a grip region 110. The outer member 102 also includes indicia and is a color. The tampon applicator also includes an inner member 104, also referred to herein as a pusher, including an opening rim region 162, and a withdrawal cord 116 extends from the end of the pusher. The pusher is the same color as the outer member but could be a different color.

In an alternative embodiment, it is to be appreciated that the insertion region 128 may include pleats instead of petals. The insertion region 128 may include two or more pleats that converge toward the longitudinal axis.

As previously discussed, heat and/or steam and/or moisture may be applied to the one or more layers and/or the cylindrical segment to aid in forming the grip region. The tampon applicator, including at least one of the outer member and the inner member, of the present invention may have a water (moisture) content (% water in sample) from 0% to about 20%; in certain embodiments, tampon applicator of the present invention may have a water (moisture) content from about 1% to about 15%; in certain embodiments, tampon applicator of the present invention may have a water (moisture) content from about 2% to about 10%; and in certain embodiments, tampon applicator of the present invention may have a water (moisture) content from about 4% to about 10%, wherein the water content is measured by the Water Content Test Method described herein.

The pusher, also referred to as an inner member, may be similarly formed. The pusher may include one or more layers of material that are wound either convolutedly or spirally. One or more coatings may be applied to the pusher either prior to winding or after winding. The end regions of the pusher may be flanged, rolled, or additional material may be added to one or both of the ends to precent the pusher from traversing through the outer member and/or to allow for ease of consumer use by signifying the end of the pusher or an increased surface area on which to engage the pusher. The pledget may be disposed within the outer member and the pusher may operatively engage the outer member by extending through the pusher opening and positioned to engage the end of pledget.

### Test Methods

### Water Content Test Method

The water (moisture) content present in a tampon applicator of the present invention is measured using the following Water Content Test Method. A tampon applicator or portion thereof (“sample”) is placed in a conditioned room at a temperature of 23° C.±1.0° C. and a relative humidity of 50%±2% for at least 24 hours prior to testing. Under the temperature and humidity conditions mentioned above, using a balance with at least four decimal places, the weight of the sample is recorded every five minutes until a change of less than 0.5% of previous weight is detected during a 10 minute period. The final weight is recorded as the “equilibrium weight”. Within 10 minutes, the samples are placed into the forced air oven on top of foil for 24 hours at 70° C.±2° C. at a relative humidity of 4%±2% for drying. After the 24 hours of drying, the sample is removed and weighed within 15 seconds. This weight is designated as the “dry weight” of the sample.

The water (moisture) content of the sample is calculated as follows:

$$\% Water in sample = 100 \% \times \frac{\begin{matrix}( Equilibrium weight of sample - \\ Dry weight of sample )\end{matrix}}{Dry weight of sample}$$

The % Water (moisture) in sample for 3 replicates is averaged to give the reported % Water (moisture) in sample. Report results to the nearest 0.1%.

### Combinations

The following combinations are contemplated herein:

A1. A method of forming a tampon applicator, the method comprising: providing a cylindrical paper segment having a longitudinal axis, an outer surface and an inner surface surrounding the longitudinal axis, wherein the leading edge portion or the trailing edge portion forms a portion of the outer surface comprising a seam; inserting a forming rod into the cylindrical paper segment along the longitudinal axis; and inverting a first end region of the cylindrical paper segment such that a portion of a wall of the first end region is adjacent to an inner surface of the cylindrical paper segment, wherein the edge of the first end region is formed about the forming rod to form a pusher opening.

A2. The method according to paragraph A1, comprising providing a paper web, wherein the paper web traverses in a machine direction; separating the paper web into discrete paper segments, wherein the discrete paper segments comprising a first side edge, a second side edge, a first surface, a second surface, a leading edge region and a trailing edge region, wherein the first side edge and the second side edge are substantially parallel to the machine direction, and wherein the leading edge region and the trailing edge region are substantially perpendicular to the machine direction; and winding the discrete paper segment to form a cylindrical paper segment.

A3. The method according to paragraph A2, comprising depositing a coating on at least a portion of the discrete paper segment.

A4. The method according to any one of the preceding paragraphs, comprising pleating a first end region of the cylindrical paper segment to form a plurality of ridges and valleys extending about the longitudinal axis, wherein at least a portion of the first end region is tapered.

A5. The method of paragraph A1, comprising holding a first portion of the forming rod with a first mandrel.

A6. The method according to paragraph A5, comprising stabilizing a second portion of the forming rod with a second mandrel.

A7. The method according to any one of the preceding paragraphs, wherein the pusher opening is non-circular.

A8. The method according to any one of the preceding paragraphs, comprising compressing the first end region to form a gripping portion, wherein the gripping portion comprises a concave surface having a radius of curvature.

A9. The method according to paragraph A8, wherein the gripping portion has a variable outer circumferential surface, and wherein the gripping portion has an area where the outer circumferential surface is smallest as measured in a plane substantially perpendicular to the longitudinal axis of the cylindrical paper segment.

A10. The method according to paragraph A9, wherein the pusher opening is positioned in the area where the outer circumferential surface is smallest.

A11. The method according to any one of the preceding paragraphs, wherein the first end region is compressed with a contact pressure of from about 25 psi to about 1000 psi to form the gripping portion.

A12. The method according to paragraph A2, wherein at least one of the first surface and the second surface of the paper web comprises an indicium.

A13. The method according to any one of the preceding paragraphs, wherein the outer surface of the cylindrical paper segment comprises an indicium.

A14. The method according to paragraph A13, wherein the indicium is continuous about the outer surface.

A15. The method according to paragraph A8, wherein the gaussian curvature of the gripping portion is from about −0.2 mm⁻² to about −0.4 mm⁻².

A16. The method according to paragraph A8, wherein heat is applied to from at least one of the pusher opening and the gripping portion.

A17. The method according to paragraph A2, wherein at least one of the first surface and the second surface comprises a coating.

A18. The method according to any one of the preceding paragraphs, wherein the pusher opening has a largest dimension of from about 3 mm to about 20 mm.

A19. The method according to any one of the preceding paragraphs, comprising inserting a pusher through the pusher opening, wherein the outer diameter of the pusher is from about 80% to about 100% of the diameter of the pusher opening.

A20. The method according to any one of the preceding paragraphs, wherein the forming rod comprises a non-circular cross-section.

A21. The method according to any one of the preceding paragraphs, comprising inserting a pusher through the pusher opening, wherein an outer perimeter of the pusher is from about 80% to about 100% of a perimeter of the pusher opening.

B22. A method of forming a tampon applicator, the method comprising: providing a cylindrical paper segment having a longitudinal axis, an outer surface and an inner surface surrounding the longitudinal axis, wherein the leading edge portion or the trailing edge portion forms a portion of the outer surface comprising a seam; inserting a first mandrel into a portion of the cylindrical paper segment; inserting a forming rod into a portion of the cylindrical paper segment along the longitudinal axis; engaging a first end region with the first mandrel; and inverting a portion of the first end region of the cylindrical paper segment such that a portion of the first end region is adjacent to an inner surface of the cylindrical paper segment, wherein the edge of the first end region is formed about the forming rod to form a pusher opening.

B23. The method according to paragraph B22, comprising cutting a portion of one or more layers of the cylindrical paper segment to form a cutout.

B24. The method according to any one of the preceding paragraphs, comprising forming one or more petals in a second end region of the cylindrical paper segment.

C25. A method of forming a tampon applicator, the method comprising: providing a cylindrical paper segment having a longitudinal axis, an outer surface and an inner surface surrounding the longitudinal axis, wherein the leading edge portion or the trailing edge portion forms a portion of the outer surface comprising a seam; inserting a first mandrel into a portion of the cylindrical paper segment; engaging a first end region with the first mandrel; and inverting a portion of the first end region of the cylindrical paper segment such that a portion of the first end region is adjacent to an inner surface of the cylindrical paper segment, wherein the edge of the first end region forms a pusher opening.

C26. The tampon applicator according to paragraph C25, wherein the tampon applicator exhibits a water content of from 0% to about 20%.

C27. The tampon applicator according to paragraph C25, wherein the tampon applicator exhibits a water content of from 1% to about 15%.

C28. The tampon applicator according to paragraph C25, wherein the tampon applicator exhibits a water content of from 4% to about 10%.

D29. A method of forming a tampon applicator, the method comprising: providing a cylindrical paper segment having a longitudinal axis, an outer surface and an inner surface surrounding the longitudinal axis, wherein the leading edge portion or the trailing edge portion forms a portion of the outer surface comprising a seam; inserting a first mandrel into the cylindrical paper segment along the longitudinal axis; and inverting a first end region of the cylindrical paper segment such that a portion of a wall of the first end region is adjacent to an inner surface of the cylindrical paper segment.

D30. The method according to paragraph D29, comprising forming a pusher opening using a forming rod.

D31. The method according to paragraph D29, comprising forming a pusher opening by die cutting a portion of the cylindrical paper segment.

D32. The method according to any one of the preceding paragraphs, comprising providing a paper web, wherein the paper web traverses in a machine direction; separating the paper web into discrete paper segments, wherein the discrete paper segments comprising a first side edge, a second side edge, a first surface, a second surface, a leading edge region and a trailing edge region, wherein the first side edge and the second side edge are substantially parallel to the machine direction, and wherein the leading edge region and the trailing edge region are substantially perpendicular to the machine direction; and winding the discrete paper segment to form a cylindrical paper segment.

D33. The method according to paragraph D32, comprising depositing a coating on at least a portion of the discrete paper segment.

D34. The method according to any one of the preceding paragraphs, comprising pleating a first end region of the cylindrical paper segment to form a plurality of ridges and valleys extending about the longitudinal axis, wherein at least a portion of the first end region is tapered.

D35. The method according to any one of the preceding paragraphs, comprising holding a first portion of a forming rod with the first mandrel.

D36. The method according to paragraph D35, comprising stabilizing a second portion of the forming rod with a second mandrel.

D37. The method according to paragraphs D30 or D31, wherein the pusher opening is non-circular.

D38. The method according to paragraphs D30 or D31, comprising compressing the first end region to form a gripping portion, wherein the gripping portion comprises a concave surface having a radius of curvature.

D39. The method according to paragraph D38, wherein the gripping portion has a variable outer circumferential surface, and wherein the gripping portion has an area where the outer circumferential surface is smallest as measured in a plane substantially perpendicular to the longitudinal axis of the cylindrical paper segment.

D40. The method according to paragraph D39, wherein the pusher opening is positioned in the area where the outer circumferential surface is smallest.

D41. The method according to any one of the preceding paragraphs, wherein the first end region is compressed with a contact pressure of from about 25 psi to about 1000 psi to form the gripping portion.

D42. The method according to paragraph D32, wherein at least one of the first surface and the second surface of the paper web comprises an indicium.

D43. The method according to any one of the preceding paragraphs, wherein the outer surface of the cylindrical paper segment comprises an indicium.

D44. The method according to paragraph D43, wherein the indicium is continuous about the outer surface.

D45. The method according to paragraph D38, wherein the gaussian curvature of the gripping portion is from about −0.2 mm⁻² to about −0.4 mm⁻².

D46. The method according to paragraph D38, wherein heat is applied to from at least one of the pusher opening and the gripping portion.

D47. The method according to paragraph D32, wherein at least one of the first surface and the second surface comprises a coating.

D48. The method according to paragraph D31, wherein the pusher opening has a largest dimension of from about 3 mm to about 20 mm.

D49. The method according to paragraph D30, comprising inserting a pusher through the pusher opening, wherein the outer diameter of the pusher is from about 80% to about 100% of the diameter of the pusher opening.

D50. The method according to paragraph D30, wherein the forming rod comprises a non-circular cross-section.

D51. The method according to paragraph D30, comprising inserting a pusher through the pusher opening, wherein an outer perimeter of the pusher is from about 80% to about 100% of a perimeter of the pusher opening.

D52. The method according to paragraph D47, wherein the coating is uncured when the first end region is compressed to form the gripping region.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of forming a tampon applicator, the method comprising:
providing a cylindrical paper segment having a longitudinal axis, an outer surface and an inner surface surrounding the longitudinal axis, wherein the leading edge portion or the trailing edge portion forms a portion of the outer surface comprising a seam;
inserting a forming rod into the cylindrical paper segment along the longitudinal axis; and
inverting a first end region of the cylindrical paper segment such that a portion of a wall of the first end region is adjacent to an inner surface of the cylindrical paper segment, wherein the edge of the first end region is formed about the forming rod to form a pusher opening.

2. The method of claim 1, comprising providing a paper web, wherein the paper web traverses in a machine direction;
separating the paper web into discrete paper segments, wherein the discrete paper segments comprising a first side edge, a second side edge, a first surface, a second surface, a leading edge region and a trailing edge region, wherein the first side edge and the second side edge are substantially parallel to the machine direction, and wherein the leading edge region and the trailing edge region are substantially perpendicular to the machine direction; and
winding the discrete paper segment to form a cylindrical paper segment.

3. The method of claim 2, comprising depositing a coating on at least a portion of the discrete paper segment.

4. The method of claim 1, comprising pleating a first end region of the cylindrical paper segment to form a plurality of ridges and valleys extending about the longitudinal axis, wherein at least a portion of the first end region is tapered.

5. The method of claim 1, comprising holding a first portion of the forming rod with a first mandrel, and stabilizing a second portion of the forming rod with a second mandrel.

6. The method of claim 1, comprising compressing the first end region to form a gripping portion, wherein the gripping portion comprises a concave surface having a radius of curvature.

7. The method of claim 6, wherein the gripping portion has a variable outer circumferential surface, and wherein the gripping portion has an area where the outer circumferential surface is smallest as measured in a plane substantially perpendicular to the longitudinal axis of the cylindrical paper segment.

8. The method of claim 7, wherein the pusher opening is positioned in the area where the outer circumferential surface is smallest.

9. The method of claim 1, wherein the first end region is compressed with a contact pressure of from about 25 psi to about 1000 psi to form the gripping portion.

10. The method of claim 2, wherein at least one of the first surface and the second surface of the paper web comprises a indicium.

11. The method of claim 6, wherein the gaussian curvature of the gripping portion is from about −0.2 mm⁻² to about −0.4 mm⁻².

12. The method of claim 6, wherein heat is applied to from at least one of the pusher opening and the gripping portion.

13. The method of claim 2, wherein at least one of the first surface and the second surface comprises a coating.

14. The method of claim 1, wherein the pusher opening has a largest dimension of from about 3 mm to about 20 mm.

15. The method of claim 1, comprising inserting a pusher through the pusher opening, wherein the outer diameter of the pusher is from about 80% to about 100% of the diameter of the pusher opening.

16. The method of claim 1, comprising inserting a pusher through the pusher opening, wherein an outer perimeter of the pusher is from about 80% to about 100% of a perimeter of the pusher opening.

17. A method of forming a tampon applicator, the method comprising:
providing a cylindrical paper segment having a longitudinal axis, an outer surface and an inner surface surrounding the longitudinal axis, wherein the leading edge portion or the trailing edge portion forms a portion of the outer surface comprising a seam;
inserting a first mandrel into a portion of the cylindrical paper segment;
inserting a forming rod into a portion of the cylindrical paper segment along the longitudinal axis;
engaging a first end region with the first mandrel; and
inverting a portion of the first end region of the cylindrical paper segment such that a portion of the first end region is adjacent to an inner surface of the cylindrical paper segment, wherein the edge of the first end region is formed about the forming rod to form a pusher opening.

18. The method of claim 17, comprising cutting a portion of one or more layers of the cylindrical paper segment to form a cutout.

19. The method of claim 17, comprising forming one or more petals in a second end region of the cylindrical paper segment.

20. A method of forming a tampon applicator, the method comprising:
providing a cylindrical paper segment having a longitudinal axis, an outer surface and an inner surface surrounding the longitudinal axis, wherein the leading edge portion or the trailing edge portion forms a portion of the outer surface comprising a seam;
inserting a first mandrel into a portion of the cylindrical paper segment;
engaging a first end region with the first mandrel; and
inverting a portion of the first end region of the cylindrical paper segment such that a portion of the first end region is adjacent to an inner surface of the cylindrical paper segment, wherein the edge of the first end region forms a pusher opening.

21. The tampon applicator according to claim 20, wherein the tampon applicator exhibits a water content of from 0% to about 20%.
